(19) United States
(12) Patent Application Publication
(10) Pub. No.: US 2012/0202231 A1
(43) Pub. Date: Aug. 9, 2012

(54) SYNERGISTIC BIOMARKER ASSAY OF NEUROLOGICAL CONDITION USING S-100B

(76) Inventors: Kevin Ka-wang Wang, Gainesville, FL (US); Ronald L. Hayes, Alachua, FL (US); Jackson Streeter, Alachua, FL (US)

(21) Appl. No.: 13/384,713

(22) PCT Filed: Jul. 19, 2010
(86) PCT No.: PCT/US10/42469

§ 371 Date: Apr. 23, 2012

Related U.S. Application Data
(60) Provisional application No. 61/271,135, filed on Jul. 18, 2009.

(51) Int. Cl. G01N 33/53

(52) U.S. Cl. 435/7.94

(57) ABSTRACT

Processes and assays are provided for detecting and determining the magnitude of traumatic brain injury such as that from impact or percussive trauma or stroke. The inventive assays and processes recognize a synergistic correlation between detection of S-IOOb and one or more other injury specific biomarkers.

### CROSS REFERENCE TO RELATED APPLICATIONS

This application claims priority to U.S. Provisional Application No. 61/271,135 filed Jul. 18, 2009, the entire contents of which are incorporated herein by reference.

### FIELD OF THE INVENTION

The present invention relates in general to determination of a neurological condition of an individual such as a brain injury and in particular to measuring a quantity of neuropredictive conditional biomarker of S-100β, UCH-L1, and/or GFAP, or combinations thereof to detect, diagnose, differentiate or treat the injury.

### BACKGROUND OF THE INVENTION

The field of clinical neurology remains frustrated by the recognition that secondary injury to a central nervous system tissue associated with physiologic response to the initial insult could be lessened if only the initial insult could be rapidly diagnosed or in the case of a progressive disorder before stress on central nervous system tissues reached a preselected threshold. Traumatic, ischemic, and neurotoxic chemical insult, along with generic disorders, all present the prospect of brain damage. While the diagnosis of severe forms of each of these causes of brain damage is straightforward through clinical response testing and computed tomography (CT) and magnetic resonance imaging (MRI) testing, these diagnostics have their limitations in that spectroscopic imaging is both costly and time consuming while clinical response testing of incapacitated individuals is of limited value and often precludes a nuanced diagnosis. Additionally, owing to the limitations of existing diagnostics, situations under which a subject experiences a stress to their neurological condition such that the subject often is unaware that damage has occurred or seek treatment as the subtle symptoms often quickly resolve. The lack of treatment of these mild to moderate challenges to neurologic condition of a subject can have a cumulative effect or subsequently result in a severe brain damage event which in either case has a poor clinical prognosis.

In order to overcome the limitations associated with spectroscopic and clinical response diagnosis of neurological condition, there is increasing attention on the use of biomarkers as internal indicators of change as to molecular or cellular level health condition of a subject. As detection of biomarkers uses a sample obtained from a subject and detects the biomarkers in that sample, typically cerebrospinal fluid, blood, or plasma, biomarker detection holds the prospect of inexpensive, rapid, and objective measurement of neurological condition. With the attainment of rapid and objective indicators of neurological condition allows one to determine severity of a non-normal brain condition on a scale with a degree of objectivity, predict outcome, guide therapy of the condition, as well as monitor subject responsiveness and recovery. Additionally, such information as obtained from numerous subjects allows one to gain a degree of insight into the mechanism of brain injury.

A number of biomarkers have been identified as being associated with severe traumatic brain injury as is often seen in vehicle collision and combat wounded subjects. Understanding how multiple biomarkers overlap and any correlations to injury severity remains unestablished. This lack of understanding is particularly prevalent with respect to traumatic injuries to the brain.

Analyses of a blast injury to a subject produced several inventive correlations between proteins and neuronal injury as an illustrative neurological condition. Neuronal injury is optionally the result of whole body blast, blast force to a particular portion of the body, or the result of other neuronal trauma or disease that produces detectable or differentiable levels of neuroactive biomarkers. Thus, identifying pathogenic pathways of primary blast brain injury (BBI) in reproducible experimental models is vital to the development of diagnostic algorithms for differentiating severe, moderate and mild (mTBI) from posttraumatic stress disorder (PTSD). Accordingly, a number of experimental animal models have been implemented to study mechanisms of blast wave impact and include rodents and larger animals such as sheep. However, because of the rather generic nature of blast generators used in the different studies, the data on brain injury mechanisms and putative biomarkers have been difficult to analyze and compare.

Thus, there exists a need for a process and an assay for providing improved measurement of neurological condition in TBI and in particular greater specificity for brain injury as compared to trauma to other tissues. There also exists a need for a process and an assay that is sensitive to mild or moderate forms of brain injury.

### SUMMARY OF THE INVENTION

A process of determining the magnitude of traumatic brain injury is provide including measuring a quantity a quantity of S-100β in a biological sample obtained from a subject at a first time and contemporaneously measuring a quantity of a second biomarker to determine an extent of traumatic brain injury in the subject. The use of S-100b along with a second biomarker provides unexpected synergistic determination of the presence of brain injury such as traumatic brain injury or that resulting from stroke (e.g. ischemic stroke) with high sensitivity thus allowing for diagnosis of mild injury requiring medical intervention and distinguishing the absence of injuries in subjects that do not need significant medical intervention.

In particular embodiments a second biomarker is UCH-L1, GFAP, vimentin; SBDP150, SBDP150N, SBDP150i, SBDP145, SBDP120 or MAP2. The first (e.g. S-100b) and second biomarkers, as well as additional biomarkers, are illustratively measured in the same or different biological samples obtained from the same subject. If different biological samples are used a second biological sample is illustratively obtained at the same time (e.g. within minutes) of the first biological sample, or at a time later, illustratively 24 hours or more following obtaining the first biological sample. It is appreciated that any biological sample in contact with the nervous system is operable. Illustratively, a biological sample is cerebrospinal fluid, whole blood, or a fraction of whole blood. A fraction of whole blood includes serum, platelet rich plasma, platelet poor plasma, or other blood fraction recognized in the art.

To further determine the magnitude of traumatic brain injury in the subject the quantity of S-100β in subject is compared to the quantity of S-100β in biological samples from other individuals with no known traumatic brain injury. The quantity of S-100b and the second or additional biomarker quantities are optionally correlated with CT scan normality or GCS score. Overall, the process allows detection of the magnitude of brain injury is no traumatic brain injury, mild traumatic brain injury, moderate traumatic brain injury, or severe traumatic brain injury.

As a means of treating TBI, or as a means of determining whether a compound has an unwanted or wanted side effect of inducing characteristic injury of TBI, one or more compounds are optionally administered prior to or following detection or determination of the magnitude of TBI.

In particular embodiments the quantity of three biomarkers are measured to determine the magnitude of TBI in a subject. Among the three biomarkers is S-100b along with two other biomarkers. A second or a third biomarker is optionally UCH-L1, GFAP, vimentin; SBDP150, SBDP150N, SBDP150i, SBDP145, SBDP120 or MAP2. In particular embodiments the three biomarkers are S-100b, UCH-L1, and GFAP. All three biomarkers are measured in one or more biological samples taken at the same time, are the same sample, or are samples taken at different time such as a later sample as described herein. The inventive process is optionally performed by measuring the quantity of S-100b, and two other biomarkers (e.g. UCH-L1 and GFAP) at the same time either in the same assay substrate or in different assay substrates. It is appreciated that one, two, or all three biomarkers are compared to the quantities of the biomarkers in the same biological sample type obtained from other individuals with no known traumatic brain injury. Also, S-100b, UCH-L1, and GFAP, for example, are correlated with CT scan normality or GCS score.

An assay is also provided including a substrate for holding a sample isolated from the subject, a S-100β specifically binding agent, a second biomarker specifically binding agent, and optionally a third biomarker specifically binding agent, whereby positively reacting said S-100β specifically binding agent and said second biomarker specific binding agent, and optionally the third biomarker specifically binding agent with a portion of the biological sample is evidence of the magnitude of the traumatic brain injury of the subject. Positively reacting is detecting the presence of a biomarker or the presence of an altered quantity of biomarker in the biological sample relative to a normal or control.

A biomarker specifically binding agent, including an S-100β specifically binding agent is optionally an antibody. The second or third biomarkers recognized by the respective biomarker specifically binding agents are optionally UCH-L1, GFAP, vimentin; SBDP150, SBDP150N, SBDP150i, SBDP145, SBDP120 or MAP2.

### BRIEF DESCRIPTION OF THE DRAWINGS

FIG. 1 represents a prior art relationship between outcome of TBI and S-100b levels in serum data taken from Raabe and Seifert Neurosurg. Rev. (2000), 23, 3, 136-138;

FIG. 2 represents the levels of UCH-L1 (A) and GFAP (B) concentration for controls and individuals in a mild/moderate traumatic brain injury cohort as determined by CT scan in samples taken upon admission and 24 hours thereafter;

FIG. 3 illustrates the concentration of UCH-L1 and GFAP as well as S100β, provided as a function of injury magnitude between control, mild, and moderate traumatic brain injury;

FIG. 4 illustrates the concentration of the same markers as depicted in FIG. 3 with respect to initial evidence upon hospital admission as to lesions in tomography scans;

FIG. 5 represents biomarkers in CSF and serum samples from the single human subject of traumatic brain injury of in human control and severe TBI human subjects as a function of time;

FIG. 6 illustrates UCH-L1 and GFAP in human control and severe TBI human subjects;

FIG. 7 illustrates UCH-L1 levels in rat CSF (A) and plasma (B) as measured by ELISA following experimental blast-induced non-penetrating injury;

FIG. 8 illustrates GFAP levels in rat CSF (A) and serum (B) as measured by ELISA following experimental blast-induced non-penetrating injury;

FIG. 9 represents UCH-L1 levels in serum following sham, mild MCAO challenge, and severe MCAO challenge;

FIG. 10 illustrates SBDP145 levels in CSF (A) and serum (B) following sham, mild MCAO challenge, and severe MCAO challenge;

FIG. 11 illustrates SBDP120 levels in CSF (A) and serum (B) following sham, mild MCAO challenge, and severe MCAO challenge;

FIG. 12 illustrates UCH-L1 levels in plasma obtained from human patients suffering ischemic or hemorrhagic stroke;

FIG. 13 illustrates levels of SBDP145 (A), SBDP120 (B), and MAP-2 (C) in plasma obtained from human patients suffering ischemic or hemorrhagic stroke;

FIG. 14 illustrates the diagnostic utility of UCH-L1 for stroke;

FIG. 15 illustrates vimentin levels is CSF from humans at various times following TBI;

FIG. 16 illustrates that with mild TBI first enrollment serum samples (N=28-29) versus normal control serum (N=173-184), UCH-L1 & S100B ROC (AUC 9.9156) is better than UCH-L1 ROC (AUC 0.8238) with p=0.0113. Similarly, GFAP & S100B ROC (AUC 0.9482) is better than GFAP ROC (AUC 0.9073) with P=0.0696;

FIG. 17 illustrates that with mild TBI (MTBI)<=48 hr serum samples (N=74-80) versus normal control serum (N=173-84), ROC(S100B & UCH-L1) AUC is 0.8418, which is larger than AUC for ROC(S100B alone)=0.8413, and ROC(UCH-L1 alone)=0.7905. GFAP & S100B ROC (AUC 0.9640) is better than GFAP ROC (AUC 0.9431) with P=0.0462. GFAP & S100B ROC (AUC 0.9640) is better than S100B ROC (AUC 0.8377) with P<0.0001;

FIG. 18 illustrates that mTBI up to 48 h serum samples (CT positive (N=14); CT negative (N=59)). ROC(S100B & UCH-L1) AUC is 0.7385, which is larger than AUC for ROC(S100B alone)=0.7222, and ROC(UCH-L1 alone)=0.6005. GFAP & S100B ROC (AUC 0.8357) is larger than GFAP ROC (AUC 0.7113) with P=0.0755. GFAP & S100B ROC (AUC 0.8357) is also better than S100B ROC (AUC 0.7054) with P=0.0544.

FIG. 19 illustrates (A) shows that first enrolment mTBI samples (N=27-72) vs. normal controls (N=173). ROC(S100B & GFAP & UCH-L1) has the largest area under the curve (AUC=0.9450). ROC(S100B & GFAP) AUC is 0.9443, which is larger than ACU for ROC(S100B alone)=0.9136, and ROC(GFAP alone)=0.9004. Similarly, ROC(S100B & UCH-L1) AUC is 0.9114, which is larger than ACU for ROC(S100B alone)=0.9136, and ROC(UCH-L1 alone)=0.8195; and (B) that ROC(S100B & GFAP & UCH-L1)_has the largest area under the curve (AUC=0.9622). ROC(S100B & GFAP) AUC is 0.9599, which is larger than ACU for ROC(S100B alone)=0.8356, and ROC(GFAP alone)=0.9367. Similarly, ROC(S100B & UCH-L1) AUC is 0.8365, which is larger than ACU for ROC(S100B alone)=0.8356, and ROC(UCH-L1 alone)=0.7848;

FIG. 20 illustrates that with mTBI first 1 h samples (CT Positive (N=14) vs. Negative N=57)): ROC(S100B & GFAP & UCH-L1) has the largest area under the curve (AUC=0.8471). ROC(S100B & GFAP) AUC is 0.8421, which is larger than ACU for ROC(S100B alone)=0.7137, and ROC(GFAP alone)=0.7249. Similarly, ROC(S100B & UCH-L1) AUC is 0.7331, which is larger than ACU for ROC(S100B alone)=0.7137, and ROC(UCH-L1 alone)=0.5915;

FIG. 21 illustrates that that MTBI first 12 hour samples: ROC for mTBI CT+(Head CT abnormal) (N=14) vs CT− (head CT negative) (N=56-57). ROC(S100B & UCH-L1) AUC is 0.7296, which is larger than ACU for ROC(S100B alone)=0.7200, and ROC(UCH-L1 alone)=0.6250. Also ROC(S100B & GFAP) AUC is 0.8411, which is larger than ACU for ROC(S100B alone)=0.7014 and ROC(GFAP alone)=0.7260;

FIG. 22 is a schematic process;

FIG. 23 is a schematic process for detecting biomarker multimers; and

FIG. 24 is a schematic process for detecting biomarker complexes.

### DESCRIPTION OF THE INVENTION

The present invention has utility in the diagnosis and management of traumatic brain injury (TBI). The subject invention also has utility as a means of detecting neurological trauma such as is the result of percussive or impact injuries or those resulting from ischemias, or disease. Through the measurement of the high specificity neuroactive biomarker UCH-L1 from a subject in combination with values obtained from the high sensitivity-low neuroactive selectivity neuroactive biomarker S-100β, a determination of subject neurological condition is provided with greater specificity as to the presence of TBI and the degree of TBI. The severity of TBI is defined based on the Glasgow scale and spans a spectrum from severe through moderate to mild.

S-100β has been found be a reliable marker of brain damage in TBI 24 h after trauma and thereafter in subjects without multiple additional traumas. S-100β is found at a high concentration in glial and Schwann cells, as well as in melanocytes, adipocytes, chondrocytes epidermal Langerhans cells, skeletal muscle, and bone marrow. S-100β does not appear to be specific for brain injury, as trauma of muscle, fat, and bone marrow all release high amounts of S-100β, and values in trauma without head injury are also increased.

While S-100β has desirable sensitivity properties as a biomarker, the lack of selectivity of S-100β towards brain trauma has proven to limit prior utility of this biomarker. As neural trauma often involves trauma to other tissues known to release S-100β there was an appreciable false positive rate resulting in unnecessary treatments for TBI. Raabe and Seifert (Neurosurg. Rev. (2000), 23, 3, 136-138), incorporated herein by reference in its entirety, illustrated a correlation between S-100β protein in serum as a marker of brain cell damage after severe head injury with injury outcome.

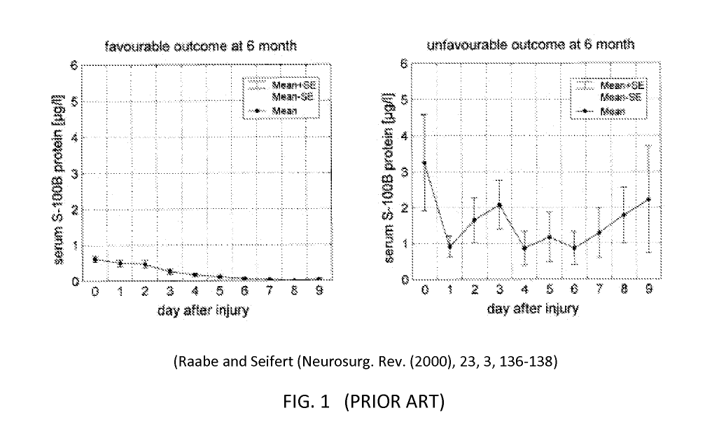

Evaluation of S-100β as a marker of injury severity is accomplished by obtaining venous blood samples after admission and every 24 hours thereafter, illustratively for 10 days. Outcome is assessed at 6 months using the Glasgow Outcome Scale. With respect to severe TBI, levels of S-100β are significantly higher in patients with unfavorable outcome compared to those with favorable outcome. (FIG. 1) (See Raabe and Seifert Neurosurg. Rev. (2000), 23, 3, 136-138, the contents of which are incorporated herein by reference.) In patients with favorable outcome, slightly increased initial levels of S-100β return to normal within 3 to 4 days. However, in patients with unfavorable outcome, initial levels are markedly increased, with a tendency to decrease from day 1 to day 6. After day 6, there tends to be a secondary increase in serum S-100β, indicating secondary brain cell damage. As such, S-100β is reliable in clinical severe TBI for which outcomes are poor. No correlative increase in S-100β has been previously observed in the absence of severe TBI. In contrast to severe injuries which are relatively easy to diagnose, minor head injury is usually defined as a clinical state involving a Glasgow Coma Scale (GCS) score of 13-15; the lower the score the more severe the injury. In contrast to prior art attempts at using S-100β as a standalone biomarker, the inventors surprisingly discovered that its detection at modestly elevated levels in combination with increases or absence thereof a second biomarker synergistically allows one to distinguish and diagnose mild and moderate forms of traumatic brain injury allowing a physician to determine which subjects are more likely to require intensive therapy. As such, a first biomarker as used herein is illustratively S-10013.

UCH-L1 (neuronal cell body damage marker) has a high degree of specificity for trauma that if measured in conjunction with S-100β provides more meaningful clinical information as to the nature and extent of the injury involved than the mere measure of S-100β alone. The nature of the UCH-L1 biomarker is detailed in U.S. Pat. Nos. 7,291,710 and 7,396,654, the contents of which are hereby incorporated by reference.

ELISA performance parameters for S-100β and UCH-L1 shown in Table 1 make clear that a synergistic value is obtained by the contemporaneous measurement of both markers. The concentration range refers to the clinically relevant concentrations and the LLD is the lower limit of detection for the ELISA assays.

TABLE 1
ELISA Performance Parameters

| protein | concentration | LLD |
|---|---|---|
| biomarker | (ng/mL) | (ng/mL) |
| UCH-L1 | 0.05-20 | 0.075 |
| S-100β^{†} | 0.01-2.0 | 0.02 |

It is appreciated that S-100β is a synergistic biomarker when used in combination with one or more additional biomarkers. Illustratively, the quantity of a second biomarker is determined in the same sample or in a second biological sample obtained at the same time, at an earlier time, or at a later time than that when the first biological sample was obtained. A second biomarker is illustratively UCH-L1; GFAP; vimentin; an SBDP illustratively 150, 150N, 150i, 145 and 120; MAP2; or additional combinations thereof. In some embodiments three biomarkers are detected including S-100b, a second biomarker, and a third biomarker. A third biomarker is illustratively UCH-L1; GFAP; vimentin; an SBDP illustratively 150, 150N, 150i, 145 and 120; or MAP2. It is appreciated that when a third biomarker is present that it is a different biomarker than a first biomarker or a second biomarker. A second biomarker and a third biomarker are not S-100b. A difference is a different protein, a different cleavage product, a different dimerization state, or a different modification such as but not limited to phosphorylation state, glycosylation state, or other recognized modification.

The recognition of the above combinations as novel and unexpectedly powerful biomarkers for neuronal injury such as TBI or stroke reveals the importance of several associations identified by the inventors between these biomarkers as illustrated in Table 2.

TABLE 2

| Novel Neural injury | |
|---|---|
| and neurological | |
| condition diagnostic | |
| biomarker pairing/ | |
| panel | Information |
| S100b + UCH-L1 | S100b (glia)-UCH-L1 (neuron) paring to |
| | monitor both neuronal and glial health and to |
| | improve diagnostic accuracy. For UCH-L1 |
| | information see Hayes et al. (2008) U.S. Pat. |
| | No. 7,396,654 B2. |
| S100b + GFAP | Both S100b and GFAP are glia protein and |
| | they co-localize subcellularly; For GFAP |
| | information see PCT-US2009-053376. |
| S100b + UCH-L1 + | S100b + GFAP + UCH-L1 triple combination |
| GFAP | improves diagnostic accuracy. |
| S100b + one of the | S100b + SBDP paring allows monitoring of both |
| alpha II-spectrin | neuronal structural (axonal) and glial health and |
| breakdown products | improves diagnostic accuracy. Alpha II-spectrin is |
| (SBDP): | an axonally enriched marker and its SBDP are |
| SBDBP150N, | produced by protease activation (calpain, caspase): |
| SBDP150, | SBDBP150N (Sequence X-QQQEVY-CO₂H), |
| SBDP145, | SBDP150 (NH₂-GMMPR-X), SBDP145 |
| SBDP150i, | (NH₂-SAHEVQR-X), SBDP150i |
| SBDP120 | (NH2-SKTASPW-X), SBDP120 (sequence |
| | NH₂-SVEAL-X); where X = 0-5 any additional |
| | amino acid. Sequence based on Human Alpha |
| | II-Spectrin II (nonerythroid) protein accession # |
| | A3571; For additional information regarding SBDP |
| | see Hayes et al. (2007) U.S. Pat. No. 7,291,710 B2 |
| S100b + MAP2 | S100b + MAP2 (neuronal dendritic marker) |
| | paring allows monitoring of both neuronal structural |
| | and glial health and improves diagnostic accuracy. |
| | See Hayes et al. (2008) U.S. Pat. No. 7,456,027 B2 |
| S100b + | Both S100b and Vimentin are glia proteins |
| Vimentin | and they co-localize subcellularly. Vimentin is a |
| | Type III filament in glia. Vimentin is a novel neural |
| | injury, neurological condition biomarker. |

In some embodiments a first biomarker is GFAP and a second biomarker is vimentin.

In some embodiments Glial Fibrillary Acidic Protein (GFAP) is detected in a biological sample along with UCH-L1 and S-100β. GFAP, as a member of the cytoskeletal protein family, is the principal 8-9 nanometer intermediate filament glial cells such as in mature astrocytes of the central nervous system (CNS). GFAP is a monomeric molecule with a molecular mass between 40 and 53 kDa and an isoelectric point between 5.7 and 5.8. GFAP is highly brain specific protein that is not found outside the CNS under normal physiological conditions. GFAP is released in response to neurological insult and released into the blood and CSF soon thereafter. In the CNS following injury, either as a result of trauma, disease, genetic disorders, or chemical insult, astrocytes become reactive in a way termed astrogliosis or gliosis that is characterized by rapid synthesis of GFAP. It is appreciated that GFAP is optionally detected as a monomer or as a multimer such as a dimer.

As used herein S-100β refers to all S100 dimers that contain a b monomer subunit, and therefore, detects the b-subunit as summed concentrations of at least 2 subtypes namely, S100BB (bb-homodimers) and S100A1-B (ab-heterodimers). It is further appreciated that S-100β refers to all 5100 monomers. Similarly, GFAP and UCH-L1 dimers are specifically included as biomarkers. While multimer formation of several biomarkers has been previously recognized, the presence of multimer formation related to diagnostic utility or other biomarker uses has not been recognized in any biofluid. For example, dimerization of S-100b, GFAP, or UCH-L1 reveal unexpected utility as differentiable biomarkers for severity of ischemic stroke or traumatic brain injury among other neuronal conditions. For additional information regarding particular pairings of biomarkers including homomultimeric pairings see Table 3.

TABLE 3

| Novel Neural injury and neurological | | |
|---|---|---|
| condition diagnostic biomarker | Information | Illustrative Detection |
| S100b-dimer | S100b complexes with | To detect S100b dimer only and |
| | itself as to form dimers. | not S100b monomer or S100b- |
| | See Garbuglia et al.. Braz J | S100a dimer, an antibody to the |
| | Med Biol Res. 1999 | same narrow epitope (Narrow is |
| | 32(10): 1177. | defined herein as less than or |
| | | equal to 10 residues, optionally |
| | | less than 6 residues, optionally 5 |
| | | residues) on the S100b protein |
| | | twice - both as capture and as |
| | | detection antibody |
| GFAP-dimer: | GFAP may exist as dimer. | To detect GFAP dimer only and |
| | See Garbuglia et al.. Braz J | not monomer, the same |
| | Med Biol Res. 1999 | antibody to the same narrow |
| | 32(10): 1177. | epitope on the GFAP protein is |
| | | used as both as capture and as |
| | | detection antibody. |
| UCH-L1-dimer | UCH-L1 exists as dimer. | To detect UCH-L1 dimer only |
| | See Bheda et al. Cell | and not monomer, the same |
| | Cycle. 2010 Mar; 9(5): 980. | antibody to the same narrow |
| | | epitope on the UCH-L1 protein is |
| | | used both as capture and as |
| | | detection antibody. |
| Vimentin as novel marker | Vimentin as a glial injury | To detect Vimentin using a |
| | marker may exist as dimer | sandwich ELISA, two vimentin |
| | or a monomer. See | antibodies are employed. |
| | Garbuglia et al.. Braz J | |
| | Med Biol Res. 1999 | |
| | 32(10): 1177. | |
| S100b-GFAP Complex | S100b and GFAP may exist | Complex as used herein |
| | as stable complex. See | illustratively means that two or |
| | Sorci et al. Biochim Biophys | more proteins associate with |
| | Acta. 1998 1448(2): 277; | each other, which is different |
| | Bianchi et al. J. Biol Chem. | from using two independent |
| | 1993 Jun | markers detected by ELISA as |
| | 15; 268(17): 12669. | pair. To detect this complex one |
| | | antibody (e.g. capture Ab) to one |
| | | protein (e.g. S100b), and a |
| | | second antibody (e.g. detection |
| | | Ab) to the other protein (e.g. |
| | | GFAP) are employed. |
| GFAP-Vimentin-complex | Vimentin and GFAP may | To detect this complex one |
| | exist as a stable complex. | antibody (e.g. capture Ab) to one |
| | See Wilhelmsson et al. J | protein (e.g. vimentin), and a |
| | Neurosci. 2004 | second antibody (e.g. detection |
| | 24(21): 5016; Lopez-Egido | Ab) to the other protein (e.g. |
| | Exp Cell Res. 2002 Aug | GFAP) are employed. |
| | 15; 278(2): 175; Jing et al.. J | |
| | Cell Sci. 2007; 120(Pt | |
| | 7): 1267. | |
| S100b--Vimentin-complex | S100b and vimentin may | To detect this complex one |
| | exist as stable complex. | antibody (e.g. capture Ab) to one |
| | See Garbuglia et al.. Braz J | protein (e.g. S100b), and a |
| | Med Biol Res. 1999 | second antibody (e.g. detection |
| | 32(10): 1177 | Ab) to the other protein (e.g. |
| | | vimentin) are employed. |

With respect to all dimers, numerous methods are known in the art for detecting multimers. These illustratively include ELISA, western blot such as under non-reducing conditions or native conditions, size exclusion chromotography, sedimentation, in situ Proximity Ligation Assay, among others known in the art and applicable herein.

Any subject that expresses an inventive biomarker is operable herein. Illustrative examples of a subject include a dog, a cat, a horse, a cow, a pig, a sheep, a goat, a chicken, non-human primate, a human, a rat, a mouse, and a cell. Subjects who benefit from the present invention are illustratively those suspected of having or at risk for developing abnormal neurological conditions, such as victims of brain injury caused by traumatic insults (e.g., gunshot wounds, automobile accidents, sports accidents, shaken baby syndrome), and ischemic events (e.g., stroke, cerebral hemorrhage, cardiac arrest).

The inventive neuroactive biomarker analyses of S-100β and one or more additional biomarkers are illustratively operable to detect and diagnose TBI of all degrees from severe to mild, owing to the specificity of a second or third biomarker and the higher degree of sensitivity associated with S-100β.

In vivo or in vitro screening or assay protocols illustratively include measurement of a neuroactive biomarker in a biological sample obtained from a subject.

Studies to determine or monitor levels of neuroactive biomarker levels of S-100b and one or more additional biomarkers are optionally combined with behavioral analyses or motor deficit analyses such as: motor coordination tests illustratively including Rotarod, beam walk test, gait analysis, grid test, hanging test and string test; sedation tests illustratively including those detecting spontaneous locomotor activity in the open-field test; sensitivity tests for allodynia—cold bath tests, hot plate tests at 38° C. and Von Frey tests; sensitivity tests for hyperalgesia—hot plate tests at 52° C. and Randall-Sellito tests; and EMG evaluations such as sensory and motor nerve conduction, Compound Muscle Action Potential (CMAP) and h-wave reflex.

An exemplary process for detecting the presence or absence of S-100β and a second biomarker in one or more biological samples involves obtaining a biological sample from a subject, such as a human, contacting the biological sample with an agent capable of detecting of the marker being analyzed, illustratively including an antibody or aptamer, and analyzing binding of the agent optionally after washing. Those samples having specifically bound agent (or reduced levels thereof in a competitive assay) express the marker being analyzed.

To provide correlations between neurological condition and measured quantities of S-100β and one or more additional biomarkers, samples of CSF or serum are collected from subjects with the samples being subjected to measurement of S-100β and one or more additional biomarkers The subjects vary in neurological condition. Detected levels of biomarkers are then optionally correlated with CT scan results as well as GCS scoring. Based on these results, an inventive assay is developed and validated such as by the methods of Lee et al., Pharmacological Research 23:312-328, 2006, the contents of which are incorporated herein by reference. It is appreciated that levels of biomarkers are obtained from one or more of many different types of biological sample. Neuroactive biomarker levels in addition to being obtained from biological samples such as CSF and serum, are also readily obtained from blood, plasma, saliva, urine, as well as solid tissue biopsy. While CSF is a commonly used sampling fluid owing to direct contact with the nervous system, it is appreciated that other biological fluids have advantages in being sampled for the same or other purposes and therefore allow for inventive determination of neurological condition optionally as part of a battery of tests performed on a single biological sample such as blood, plasma, serum, saliva or urine.

A biological sample is obtained from a subject by conventional techniques. For example, CSF is obtained by lumbar puncture. Blood is obtained by venipuncture, while plasma and serum are obtained by fractionating whole blood according to known methods. Surgical techniques for obtaining solid tissue samples are well known in the art. For example, methods for obtaining a nervous system tissue sample are described in standard neurosurgery texts such as Atlas of Neurosurgery: Basic Approaches to Cranial and Vascular Procedures, by F. Meyer, Churchill Livingstone, 1999; Stereotactic and Image Directed Surgery of Brain Tumors, 1st ed., by David G. T. Thomas, WB Saunders Co., 1993; and Cranial Microsurgery: Approaches and Techniques, by L. N. Sekhar and E. De Oliveira, 1st ed., Thieme Medical Publishing, 1999, the contents of each of which are incorporated herein by reference. Methods for obtaining and analyzing brain tissue are also described in Belay et al., Arch. Neurol. 58: 1673-1678 (2001); and Seijo et al., J. Clin. Microbiol. 38: 3892-3895 (2000), the contents of which are incorporated herein by reference.

A process as provided herein can be used to detect S-100β and one or more additional biomarkers in a biological sample in vitro, as well as in vivo. The quantity of expression of S-100β and one or more additional biomarkers in a sample is optionally compared with appropriate controls such as a first sample known to express detectable levels of the marker being analyzed (positive control) and/or a second sample known to not express detectable levels of the marker being analyzed (a negative control). For example, in vitro techniques for detection of a marker include enzyme linked immunosorbent assays (ELISAs), western blots, immunoprecipitation, and immunofluorescence. Also, in vivo techniques for detection of a marker illustratively include introducing a labeled agent that specifically binds the marker into a biological sample or test subject. For example, the agent can be labeled with a radioactive marker whose presence and location in a biological sample or test subject can be detected by standard imaging techniques.

Any suitable molecule that can specifically binds S-100β or one or more additional biomarkers or any suitable molecule that specifically binds one or more other neuroactive biomarkers are operative in the invention to achieve a synergistic assay. An exemplary agent for biomarker detection and quantification is an antibody capable of binding to the biomarker being analyzed. An antibody is optionally conjugated to a detectable label. Such antibodies can be polyclonal or monoclonal. An intact antibody, a fragment thereof (e.g., Fab or F(ab′)₂), or an engineered variant thereof (e.g., sFv) can also be used. Such antibodies can be of any immunoglobulin class including IgG, IgM, IgE, IgA, IgD and any subclass thereof.

Antibody-based assays are illustratively used analyzing a biological sample for the presence of biomarker. Suitable western blotting methods are optionally used. For more rapid analysis (as may be important in emergency medical situations), immunosorbent assays (e.g., ELISA and RIA) and immunoprecipitation assays may be used. As one example, the biological sample or a portion thereof is immobilized on a substrate, such as a membrane made of nitrocellulose or PVDF; or a rigid substrate made of polystyrene or other plastic polymer such as a microtiter plate, and the substrate is contacted with an antibody that specifically binds a second or additional biomarker and a second antibody specific for S-100β under conditions that allow binding of antibody to the biomarker being analyzed. After washing, the presence of the antibody on the substrate indicates that the sample contained the marker being assessed. If the antibody is directly conjugated with a detectable label, such as an enzyme, fluorophore, or radioisotope, the label presence is optionally detected by examining the substrate for the detectable label. Alternatively, a detectably labeled secondary antibody that binds the marker-specific antibody is added to the substrate. The presence of detectable label on the substrate after washing indicates that the sample contained the marker.

Numerous permutations of these basic immunoassays are also operative in the invention. These include the biomarker-specific antibody, as opposed to the sample being immobilized on a substrate, and the substrate is contacted with biomarker conjugated with a detectable label under conditions that cause binding of antibody to the labeled marker. The substrate is then contacted with a sample under conditions that allow binding of the marker being analyzed to the antibody. A reduction in the amount of detectable label on the substrate after washing indicates that the sample contained the marker.

Although antibodies are preferred for use in the invention because of their extensive characterization, any other suitable agent (e.g., a peptide, an aptamer, or a small organic molecule) that specifically binds a biomarker is optionally used in place of the antibody in the above-described immunoassays. Aptamers are nucleic acid-based molecules that bind specific ligands. Methods for making aptamers with a particular binding specificity are known as detailed in U.S. Pat. Nos. 5,475,096; 5,670,637; 5,696,249; 5,270,163; 5,707,796; 5,595,877; 5,660,985; 5,567,588; 5,683,867; 5,637,459; and 6,011,020, the contents of each of which are incorporated herein by reference.

A myriad of detectable labels are operative in a diagnostic assay for biomarker expression and are known in the art. Labels and labeling kits are commercially available optionally from Invitrogen Corp, Carlsbad, Calif. Agents used in methods for detecting a neuroactive biomarker are optionally conjugated to a detectable label, e.g., an enzyme such as horseradish peroxidase. Agents labeled with horseradish peroxidase can be detected by adding an appropriate substrate that produces a color change in the presence of horseradish peroxidase. Several other detectable labels that may be used are known. Common examples include alkaline phosphatase, horseradish peroxidase, fluorescent molecules, luminescent molecules, colloidal gold, magnetic particles, biotin, radioisotopes, and other enzymes.

The present invention employs a step of correlating the presence or amount of S-100β and one or more additional biomarkers in a biological sample with the severity and/or type of TBI. The amount of UCH-L1, for example, and S-100β in the biological sample is associated with neurological condition for traumatic brain injury such as by methods detailed in the examples. The results of an inventive assay to synergistically measure S-100β and one or more additional biomarkers can help a physician, veterinarian, or scientist determine the type and severity of injury with implications as to the types of cells that have been compromised. These results are in agreement with CT scan and GCS results, yet are quantitative, obtained more rapidly, and at far lower cost.

An assay or process optionally provides a step of comparing the quantity of S-100β and one or more additional biomarkers to normal levels of one or each to determine the neurological condition of the subject. The practice of an inventive process provides a test which can help a physician determine suitable therapeutics to administer for optimal benefit of the subject.

An assay for analyzing cell damage in a subject is also provided. The assay includes: (a) a substrate for holding a sample isolated from a subject suspected of having a damaged nerve cell, the sample being a fluid in communication with the nervous system of the subject prior to being isolated from the subject; (b) a S-100β specific binding agent specific binding agent; (c) a second biomarker specific binding agent; and optionally (d) printed instructions for reacting: the second biomarker specific agent with the biological sample or a portion of the biological sample to detect the presence or amount of the second biomarker, and the agent specific for S-100β with the biological sample or a portion of the biological sample to detect the presence or amount of S-100β and the second biomarker in the biological sample. The inventive assay can be used to detect neurological condition for financial renumeration. In some embodiments a third biomarker specific agent is included that is specific for a third biomarker that is different than a second biomarker and is not S-100b.

Baseline levels of biomarkers are those levels obtained in the target biological sample in the species of desired subject in the absence of a known neurological condition. These levels need not be expressed in hard concentrations, but may instead be known from parallel control experiments and expressed in terms of fluorescent units, density units, and the like. Typically, in the absence of a neurological condition, one or more biomarkers are present in biological samples at a negligible amount. However, UCH-L1 is a highly abundant protein in neurons. Determining the baseline levels of biomarkers illustratively including UCH-L1 or S100β protein as well as RNA in neurons, plasma, or CSF, for example, of particular species is well within the skill of the art. Similarly, determining the concentration of baseline levels of other biomarkers is well within the skill of the art. Baseline levels are illustratively the quantity or activity of a biomarker in a sample from one or more subjects that are not suspected of having a neurological condition.

The relative levels of S-100b or one or more additional biomarkers are optionally expressed as a ratio to control, baseline, or known elevated biomarker levels. As used herein a “ratio” is either a positive ratio wherein the level of the target biomarker is greater than the target in a second sample or relative to a known or recognized baseline level of the same target. A negative ratio describes the level of the target as lower than the target in a second sample or relative to a known or recognized baseline level of the same target. A neutral ratio describes no observed change in target biomarker.

A neurological condition optionally results in or produces an injury. As used herein an “injury” is an alteration in cellular or molecular integrity, activity, level, robustness, state, or other alteration that is traceable to an event. Injury illustratively includes a physical, mechanical, chemical, biological, functional, infectious, or other modulator of cellular or molecular characteristics. An injury optionally results from an event. An event is illustratively, a physical trauma such as an impact (illustratively, percussive) or a biological abnormality such as a stroke resulting from blockade (ischemic) of a blood vessel. As such the term “traumatic brain injury” (TBI) is meant to describe injury to the brain as the result of an event such as percussion or other impact, or blockade of a blood vessel.

An injury is optionally a physical event such as a percussive impact. An impact is optionally the like of a percussive injury such as resulting to a blow to the head, the body, or combinations thereof that either leave the cranial structure intact or results in breach thereof. Experimentally, several impact methods are used illustratively including controlled cortical impact (CCI) at a 1.6 mm depression depth, equivalent to severe TBI in human. This method is described in detail by Cox, CD, et al., J Neurotrauma, 2008; 25(11):1355-65, the contents of which are incorporated herein by reference. It is appreciated that other experimental methods producing impact trauma are similarly operable.

An injury may also result from stroke. Ischemic stroke is optionally modeled by middle cerebral artery occlusion (MCAO) in rodents. UCH-L1 protein levels, for example, are increased following mild MCAO which is further increased following severe MCAO challenge. Mild MCAO challenge may result in an increase of biomarker levels within two hours that is transient and returns to control levels within 24 hours. In contrast, severe MCAO challenge results in an increase in biomarker levels within two hours following injury and may be much more persistent demonstrating statistically significant levels out to 72 hours or more.

A step of correlating the presence or amount of a biomarker in a biological sample with the severity and/or type of nerve cell (or other biomarker-expres sing cell) toxicity is optionally provided. The amount of biomarker(s) in the biological sample directly relates to severity of neurological condition as a more severe injury damages a greater number of nerve cells which in turn causes a larger amount of biomarker(s) to accumulate in the biological sample (e.g., CSF; serum). Illustratively, elevated levels of UCH-L1, GFAP, or both along with modestly elevated levels of S-100b reveal severe TBI. Elevated UCH-L1, GFAP or both along with no appreciable increase in S-100β can reveal moderate TBI. Absence of increases in S-100β and one UCH-L1, GFAP or both following an impact reveal mild TBI. Also, the level of or kinetic extent of biomarkers present in a biological sample may optionally distinguish mild injury from a more severe injury. In an illustrative example, severe MCAO (2h) produces increased UCH-L1 in both CSF and serum relative to mild challenge (30 min) while both produce UCH-L1 levels in excess of uninjured subjects. Moreover, the persistence or kinetic extent of the markers in a biological sample is indicative of the severity of the neurotoxicity with greater toxicity indicating increases persistence of UCH-L1 or S-100β biomarkers in the subject that is measured in a process in biological samples taken at several time points following injury.

The invention optionally includes administration one or more compounds such as therapeutic agents or molecules being assayed for therapeutic or other potential that may alter one or more characteristics of a target biomarker such as concentration in a biological sample. A therapeutic optionally serves as an agonist or antagonist of a target biomarker or upstream effector of a biomarker. A therapeutic optionally affects a downstream function of a biomarker. For example, Acetylcholine (Ach) plays a role in pathological neuronal excitation and TBI-induced muscarinic cholinergic receptor activation may contribute to excitotoxic processes. As such, biomarkers optionally include levels or activity of Ach or muscarinic receptors. Optionally, an operable biomarker is a molecule, protein, nucleic acid or other that is effected by the activity of muscarinic receptor(s). As such, therapeutics operable in the subject invention illustratively include those that modulate various aspects of muscarinic cholinergic receptor activation.

Specific muscarinic receptors operable as therapeutic targets or modulators of therapeutic targets include the M₁, M₂, M₃, M₄, and M₅ muscarinic receptors.

The suitability of the muscarinic cholinergic receptor pathway in detecting and treating TBI arises from studies that demonstrated elevated ACh in brain cerebrospinal fluid (CSF) following experimental TBI (Gorman et al., 1989; Lyeth et al., 1993a) and ischemia (Kumagae and Matsui, 1991), as well as the injurious nature of high levels of muscarinic cholinergic receptor activation through application of cholinomimetics (Olney et al., 1983; Turski et al., 1983). Furthermore, acute administration of muscarinic antagonists improves behavioral recovery following experimental TBI (Lyeth et al., 1988a; Lyeth et al., 1988b; Lyeth and Hayes, 1992; Lyeth et al., 1993b; Robinson et al., 1990). As such chemical or biological agents such as compounds that bind to, or alter a characteristic of a muscarinic cholinergic receptor are optionally screened for neurotoxicity of cells or tissues such as during target optimization in pre-clinical drug discovery.

A compound illustratively a therapeutic compound, chemical compound, or biological compound is illustratively any molecule, family, extract, solution, drug, pro-drug, or other that is operable for changing, optionally improving, therapeutic outcome of a subject at risk for or subjected to a neurotoxic insult. A therapeutic compound is optionally a muscarinic cholinergic receptor modulator such as an agonist or antagonist, an amphetamine. An agonist or antagonist may by direct or indirect. An indirect agonist or antagonist is optionally a molecule that breaks down or synthesizes acetylcholine or other muscarinic receptor related molecule illustratively, molecules currently used for the treatment of Alzheimer's disease. Cholinic mimetics or similar molecules are operable herein. An exemplary list of therapeutic compounds operable herein include: dicyclomine, scoplamine, milameline, N-methyl-4-piperidinylbenzilate NMP, pilocarpine, pirenzepine, acetylcholine, methacholine, carbachol, bethanechol, muscarine, oxotremorine M, oxotremorine, thapsigargin, calcium channel blockers or agonists, nicotine, xanomeline, BuTAC, clozapine, olanzapine, cevimeline, aceclidine, arecoline, tolterodine, rociverine, IQNP, indole alkaloids, himbacine, cyclostellettamines, derivatives thereof, pro-drugs thereof, and combinations thereof. A therapeutic compound is optionally a molecule operable to alter the level of or activity of a calpain or caspase. Such molecules and their administration are known in the art. It is appreciated that a compound is any molecule including molecules of less than 700 Daltons, peptides, proteins, nucleic acids, or other organic or inorganic molecules that is contacted with a subject, or portion thereof.

A compound is optionally any molecule, protein, nucleic acid, or other that alters the level of a neuroactive biomarker in a subject. A compound is optionally an experimental drug being examined in pre-clinical or clinical trials, or is a compound whose characteristics or affects are to be elucidated. A compound is optionally kainic acid, MPTP, an amphetamine, cisplatin or other chemotherapeutic compounds, antagonists of a NMDA receptor, any other compound listed herein, pro-drugs thereof, racemates thereof, isomers thereof, or combinations thereof. Example amphetamines include: ephedrine; amphetamine aspartate monohydrate; amphetamine sulfate; a dextroamphetamine, including dextroamphetamine saccharide, dextroamphetamine sulfate; methamphetamines; methylphenidate; levoamphetamine; racemates thereof; isomers thereof; derivatives thereof; or combinations thereof. Illustrative examples of antagonists of a NMDA receptor include those listed in Table 4 racemates thereof, isomers thereof, derivatives thereof, or combinations thereof:

TABLE 4

| | | |
|---|---|---|
| AP-7 (drug) | Gacyclidine | PEAQX |
| AP5 | Hodgkinsine | Perzinfotel |
| Amantadine | Huperzine A | Phencyclidine |
| Aptiganel | Ibogaine | 8A-PDHQ |
| CGP-37849 | Ifenprodil | Psychotridine |
| DCKA | Indantadol | Remacemide |
| Delucemine | Ketamine | Rhynchophylline |
| Dexanabinol | Kynurenic acid | Riluzole |
| Dextromethorphan | Lubeluzole | Sabeluzole |
| Dextrorphan | Memantine | Selfotel |
| Dizocilpine | Midafotel | Tiletamine |
| Eliprodil | Neramexane | Xenon |
| Esketamine | Nitrous oxide | |
| Ethanol | | |
| NEFA | | |

As used herein the term “administering” is delivery of a compound to a subject. The compound is a chemical or biological agent administered with the intent to ameliorate one or more symptoms of a condition or treat a condition. A therapeutic compound is administered by a route determined to be appropriate for a particular subject by one skilled in the art. For example, the therapeutic compound is administered orally, parenterally (for example, intravenously, by intramuscular injection, by intraperitoneal injection, intratumorally, by inhalation, or transdermally. The exact amount of therapeutic compound required will vary from subject to subject, depending on the age, weight and general condition of the subject, the severity of the neurological condition that is being treated, the particular therapeutic compound used, its mode of administration, and the like. An appropriate amount may be determined by one of ordinary skill in the art using only routine experimentation given the teachings herein or by knowledge in the art without undue experimentation.

Processes of detecting or distinguishing the magnitude of traumatic brain injury (TBI) is also provided. Traumatic brain injury is illustratively mild-TBI, moderate-TBI, or severe-TBI. As used herein mild-TBI is defined as individuals presenting with a CGS score of 12-15 or any characteristic described in the National Center for Injury Prevention and Control, Report to Congress on Mild Traumatic Brain Injury in the United States: Steps to Prevent a Serious Public Health Problem. Atlanta, Ga.: Centers for Disease Control and Prevention; 2003, incorporated herein by reference. Moderate-TBI is defined as presenting a GCS score of 9-11. Severe-TBI is defined as presenting a GCS score of less than 9, presenting with an abnormal CT scan or by symptoms including unconsciousness for more than 30 minutes, post traumatic amnesia lasting more than 24 hours, and penetrating cranialcerebral injury.

A process of detecting or distinguishing between mild- or moderate-TBI illustratively includes obtaining a sample from a subject at a first time and measuring a quantity of S-100β and a second biomarker in the sample where an elevated S-100β and second biomarker level indicates the presence of traumatic brain injury. The inventive process is optionally furthered by correlating the quantity of S-100β and second biomarker with CT scan normality or GCS score. A positive correlation for mild-TBI is observed when the GCS score is 12 or greater, and neither S-100β nor second biomarker levels are elevated. A positive correlation for moderate-TBI is observed when the GCS score is 9-11 and second biomarker levels are elevated with modest elevation of S-100β returning to low levels within 24 hours of injury. Alternatively or in addition, a positive correlation for moderate-TBI is observed when the CT scan results are abnormal, and second biomarker levels are elevated. Abnormal CT scan results are illustratively the presence of lesions. Unremarkable or normal CT scan results are the absence of lesions.

The levels of S-100β and one or more additional biomarkers are optionally measured in samples obtained within 24 hours of injury. Illustratively, UCH-L1 and S-100β levels are measured in samples obtained 0-24 hours of injury inclusive of all time points therebetween. In some embodiments, a second sample is obtained at or beyond 24 hours following injury and the quantity of S-100β alone or along with additional biomarkers are measured.

Various aspects of the present invention are illustrated by the following non-limiting examples. The examples are for illustrative purposes and are not a limitation on any practice of the present invention. It will be understood that variations and modifications can be made without departing from the spirit and scope of the invention. While the examples are generally directed to mammalian tissue, specifically, analyses of rat tissue, a person having ordinary skill in the art recognizes that similar techniques and other techniques know in the art readily translate the examples to other mammals such as humans. Reagents illustrated herein are commonly cross reactive between mammalian species or alternative reagents with similar properties are commercially available, and a person of ordinary skill in the art readily understands where such reagents may be obtained.

### Example 1

Materials for Biomarker Analyses. Sodium bicarbonate, (Sigma Cat #: C-3041), blocking buffer (Startingblock T20-TBS) (Pierce Cat#: 37543), Tris buffered saline with Tween 20 (TBST; Sigma Cat #: T-9039). Phosphate buffered saline (PBS; Sigma Cat #: P-3813); Tween 20 (Sigma Cat #: P5927); Ultra TMB ELISA (Pierce Cat #: 34028); and Nunc maxisorp ELISA plates (Fisher). Monoclonal and polyclonal UCH-L1 antibodies are made in-house or are obtained from Santa Cruz Biotechnology, Santa Cruz, Calif. Antibodies directed to S-100β are available from Santa Cruz Biotechnology, Santa Cruz, Calif. Antibodies to GFAP are made in-house or are available from Santa Cruz Biotechnology, Santa Cruz, Calif. Labels for antibodies of numerous subtypes are available from Invitrogen, Corp., Carlsbad, Calif. Protein concentrations in biological samples are determined using bicinchoninic acid microprotein assays (Pierce Inc., Rockford, Ill., USA) with albumin standards. All other necessary reagents and materials are known to those of skill in the art and are readily ascertainable.

Biomarker specific rabbit polyclonal antibodies and monoclonal antibodies are produced in the laboratory or are available from commercial sources known to those of skill in the art. To determine reactivity specificity of the antibodies a tissue panel is probed by western blot.

An indirect ELISA is used with recombinant biomarker protein attached to the ELISA plate to determine optimal concentration of the antibodies used in the assay. This assay determines suitable concentrations of biomarker specific binding agent to use in the assay. Microplate wells are coated with rabbit polyclonal antihuman biomarker antibody. After determining concentration of rabbit antihuman biomarker antibody for a maximum signal, maximal detection limit of the indirect ELISA for each antibody is determined. An appropriate diluted sample is incubated with a rabbit polyclonal antihuman biomarker antibody (capture antibody) for 2 hours and then washed. Biotin labeled monoclonal antihuman biomarker antibody is then added and incubated with captured biomarker. After thorough wash, streptavidin horseradish peroxidase conjugate is added. After 1 hour incubation and the last washing step, the remaining conjugate is allowed to react with substrate of hydrogen peroxide tetramethyl benzadine. The reaction is stopped by addition of the acidic solution and absorbance of the resulting yellow reaction product is measured at 450 nanometers. The absorbance is proportional to the concentration of the biomarker. A standard curve is constructed by plotting absorbance values as a function of biomarker concentration using calibrator samples and concentrations of unknown samples are determined using the standard curve.

ELISA is used to more rapidly and readily detect and quantitate UCH-L1 in biological samples in rats following CCI. For a UCH-L1 sandwich ELISA (swELISA), 96-well plates are coated with 100 μL/well capture antibody (500 ng/well purified rabbit anti-UCH-L1, made in-house by conventional techniques) in 0.1 M sodium bicarbonate, pH 9.2. Plates are incubated overnight at 4° C., emptied and 300 μl/well blocking buffer (Startingblock T20-TBS) is added and incubated for 30 min at ambient temperature with gentle shaking. This is followed by either the addition of the antigen standard (recombinant UCH-L1) for standard curve (0.05-50 ng/well) or samples (3-10 μl CSF) in sample diluent (total volume 100 μl/well). The plate is incubated for 2 hours at room temperature then washed with automatic plate washer (5×300 μl/well with wash buffer, TBST). Detection antibody mouse anti-UCH-L1-HRP conjugated (made in-house, 50 μg/ml) in blocking buffer is then added to wells at 100 μL/well and incubated for 1.5 h at room temperature, followed by washing. If amplification is needed, biotinyl-tyramide solution (Perkin Elmer Elast Amplification Kit) is added for 15 min at room temperature, washed then followed by 100 μl/well streptavidin-HRP (1:500) in PBS with 0.02% Tween-20 and 1% BSA for 30 min and then followed by washing. Lastly, the wells are developed with 100 μl/well TMB substrate solution (Ultra-TMB ELISA, Pierce#34028) with incubation times of 5-30 minutes. The signal is read at 652 nm with a 96-well spectrophotometer (Molecular Device Spectramax 190). Similar assays are performed using primary antibodies directed to S-100β and UCH-L1.

To specifically detect dimers of S-100β, UCH-L1, or GFAP an ELISA assay is used where the capture and detection antibodies are directed to identical epitopes that are not involved in the dimerization of biomarker using similar techniques to those described by El-Agnaf OMA, et al, The FASEB Journal, 2006; 20:419-425, the contents of which are incorporated herein by reference. The above assay for UCH-L1 is repeated using 96-well plates coated with S-100β antibody from Santa Cruz Biotechnology and blocked with blocking buffer (Startingblock T20-TBS) as described above. Samples (100 μL/well) are incubated with the plates for 2 hours at room temperature, followed by washing with an automatic plate washer (5×300 μl/well with wash buffer, TBST). Detection antibody is the identical antibody as the primary antibody but additionally conjugated with HRP (made in-house, 50 μg/ml), placed in blocking buffer and then added to wells at 100 μL/well and incubated for 1.5 h at room temperature, followed by washing. The wells are developed with 100 μl/well TMB substrate solution (Ultra-TMB ELISA, Pierce#34028) with incubation times of 5-30 minutes. The signal is read at 652 nm with a 96-well spectrophotometer (Molecular Device Spectramax 190). The assay allows specific detection of dimers. During assay development, identical samples are subjected to size exclusion chromatography as per are recognized methods and fractions are assayed by the single antibody ELISA. Positive results in higher molecular weight protein containing fractions are indicative of biomarker dimers.

### Example 2

Severe Traumatic Brain Injury Study—46 subjects suffering severe traumatic brain injury are studied for biomarker levels in various tissues and at various times following injury. Each of these subjects is over age 18, has a GCS of less than or equal to 8, and required ventriculostomy and neuromonitoring are performed as part of routine care. Control group A, synonymously detailed as CSF controls, includes 10 individuals also being over the age of 18 or older and no injuries. Samples are obtained during spinal anesthesia for routine surgical procedures, or access to CSF is associated with treatment of hydrocephalus or meningitis. A control group B, synonymously described as normal controls, totals 64 individuals, each age 18 or older and experiencing multiple injuries without brain injury. Further details with respect to the demographics of the study are provided in Table 5.

TABLE 5
Subject Demographics for Severe Traumatic Brain Injury Study

| | | | CSF | |
|---|---|---|---|---|
| | | TBI | Controls | Normal Controls |
| Number | | 46 | 10 | 64 |
| | Males | 34 (73.9%) | 29 (65.9%) | 26 (40.6%) |
| | Females | 12 (26.1%) | 15 (34.1%) | 38 (59.4% |
| Age: | Average | 50.2 | 58.2 1, 2 | 30.09 2, 3 |
| | Std Dev | 19.54 | 20.52 | 15.42 |
| | Minimum | 19 | 23 | 18 |
| | Maximum | 88 | 82 | 74 |
| Race: | Caucasian | | | |
| | Black | 45 | 38 (86.4%) | 52 (81.2%) |
| | Asian | 1 | 6 (13.6) | 4 (6.3%) |
| | Other | | | 7 (10.9%) |
| | | | | 1 (1.6%) |
| GCS in | Average | 5.3 | | |
| Emergency | Std Dev | 1.9 | | |
| Department | | | | |

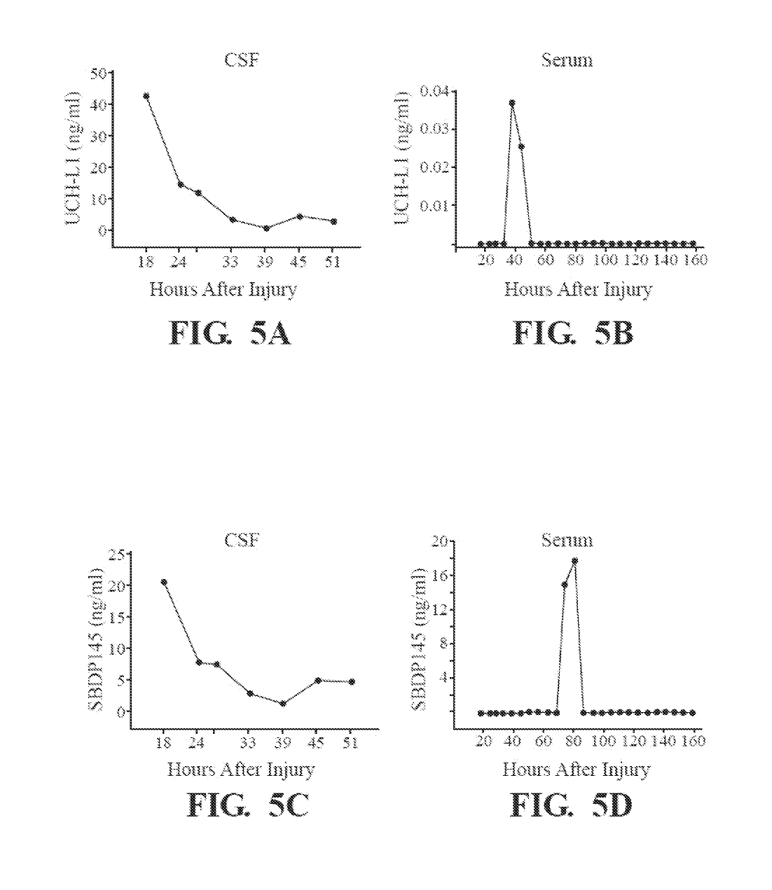

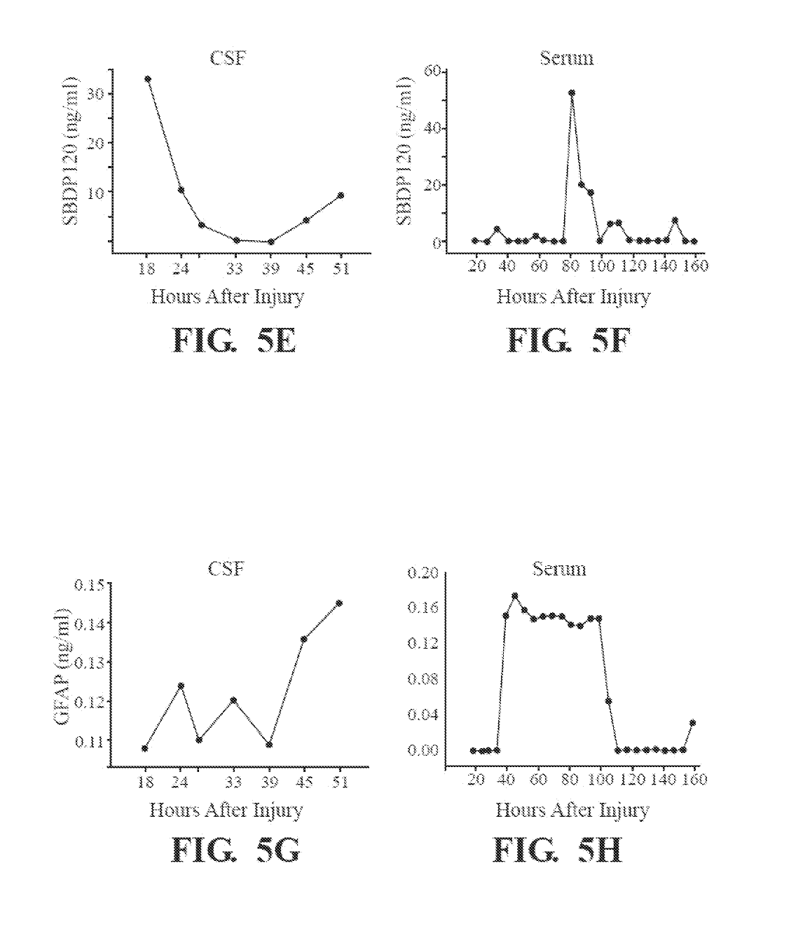

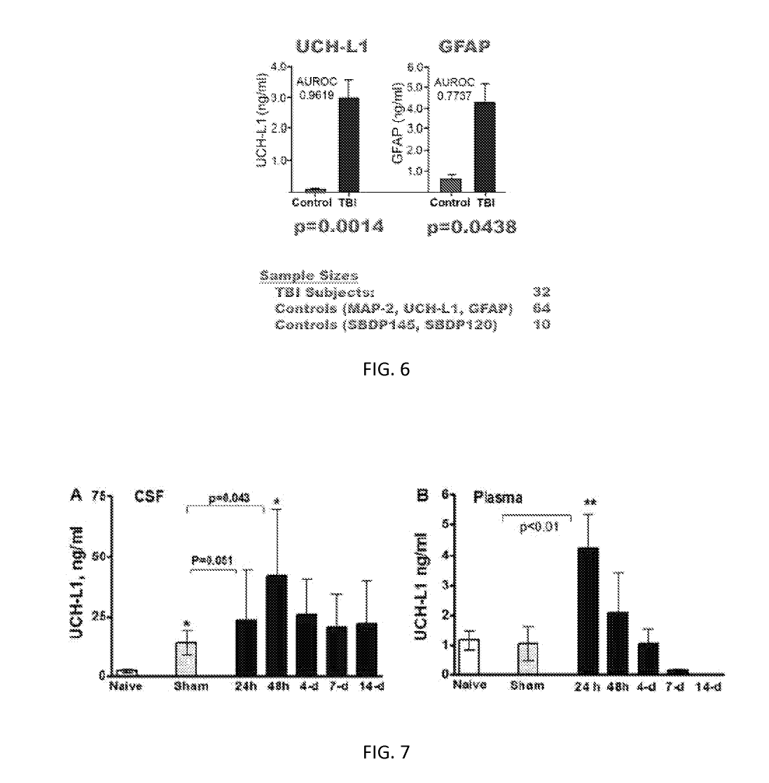

The levels of biomarkers found in the first available CSF and serum samples obtained in the study are analyzed by ELISA essentially as described in Example 1 with the recombinant biomarker replaced by sample and results are provided in FIGS. 5 and 6. The average first CSF sample collected as detailed in FIG. 6 is between 10.1 and 11.2 hours. The quantity of each of biomarkers UCH-L1 and GFAP are provided for each sample for the cohort of traumatic brain injury sufferers as compared to a control group (FIG. 6). The diagnostic utility of the various biomarkers within the first 12 hours subsequent to injury based on a compilation of CSF and serum data is provided in FIG. 6 and indicates in particular the value of GFAP as well as that of additional markers UCH-L1 and the spectrin breakdown products. Elevated levels of UCH-L1 are indicative of the compromise of neuronal cell body damage while an increase in S-100β synergistically indicates trauma.

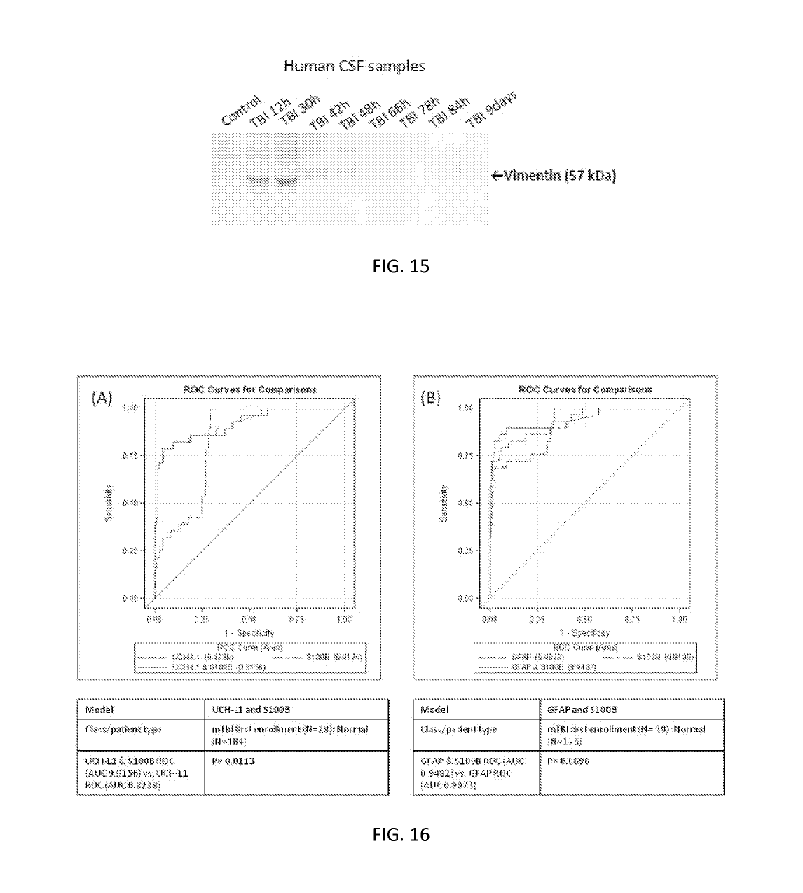

The concentration of spectrin breakdown products, GFAP, and UCH-L1 as a function of time subsequent to traumatic brain injury is illustrated in FIG. 5 and has been reported elsewhere as exemplified in U.S. Pat. Nos. 7,291,710 and 7,396,654 each of which is incorporated herein by reference. The levels of vimentin following TBI are illustrated in FIG. 15.

An analysis is performed to evaluate the ability of biomarkers measured in serum to predict TBI outcome, specifically GCS. Stepwise regression analysis is used to evaluate biomarkers as an independent predictive factor, along with the demographic factors of age and gender, and also interactions between pairs of factors. Interactions determine important predictive potential between related factors, such as when the relationship between a biomarker and outcome may be different for men and women, such a relationship would be defined as a gender by biomarker interaction.

The resulting analysis identifies biomarkers UCH-L1 and GFAP as being statistically significant predictors of GCS (Tables 6, 7). Furthermore, GFAP has improved predictability when evaluated in combination with UCH-L1 and gender (Tables 8, 9).

TABLE 6
Stepwise Regression Analysis 1 - Cohort includes:
All Subjects >= 18 Years Old
Summary of Stepwise Selection - 48 Subjects

| Variable | Parameter | Model | | |
|---|---|---|---|---|
| Step Entered | Estimate | R-Square | F Value | p-value |
| Intercept | 13.02579 | | | |
| 2 SEXCD | −2.99242 | 0.1580 | 7.29 | 0.0098 |
| 1 CSF_UCH_L1 | −0.01164 | 0.2519 | 11.54 | 0.0015 |
| 3 Serum_MAP_2 | 0.96055 | 0.3226 | 4.59 | 0.0377 |

TABLE 7
Stepwise Regression Analysis 2 - Cohort includes:
TBI Subjects >= 18 Years Old
Summary of Stepwise Selection - 39 Subjects

| Variable | Parameter | Model | | |
|---|---|---|---|---|
| Step Entered | Estimate | R-Square | F Value | p-value |
| Intercept | 5.73685 | | | |
| 1 Serum_UCH_L1 | −0.30025 | 0.0821 | 8.82 | 0.0053 |
| 2 Serum_GFAP | 0.12083 | 0.1973 | 5.16 | 0.0291 |

TABLE 8
Stepwise Regression Analysis 1 - Cohort includes:
TBI and A Subjects >= 18 Years Old
Summary of Stepwise Selection - 57 Subjects

| Variable | Parameter | Model | | |
|---|---|---|---|---|
| Step Entered | Estimate | R-Square | F Value | p-value |
| Intercept | 8.04382 | | | |
| 1 Serum_UCH_L | −0.92556 | 0.1126 | 12.90 | 0.0007 |
| 2 Serum_MAP_2 | 1.07573 | 0.2061 | 5.79 | 0.0197 |
| 3 Serum UCH-L1 + | 0.01643 | 0.2663 | 4.35 | 0.0419 |
| Serum_GFAP | | | | |

TABLE 9
Stepwise Regression Analysis 2 - Cohort includes:
TBI Subjects >= 18 Years Old
Summary of Stepwise Selection - 44 Subjects

| Variable | Parameter | Model | | |
|---|---|---|---|---|
| Step Entered | Estimate | R-Square | F Value | p-value |
| Intercept | 5.50479 | | | |
| 1 Serum_UCH_L1 | −0.36311 | 0.0737 | 11.95 | 0.0013 |
| 2 SEX_Serum_GFAP | 0.05922 | 0.1840 | 5.09 | 0.0296 |
| 3 Serum_MAP_2 | 0.63072 | 0.2336 | 2.59 | 0.1157 |

### Example 3

Mild or Moderate Traumatic Brain Injury Study. Subjects in the study of Example 2 with GCS scores too high to be qualified as having a magnitude of TBI defined as severe are further studied for biomarker levels relating to mild or moderate traumatic brain injury, as the most difficult to diagnose. Each of these subjects is characterized by being over age 18, having a GCS of between 9 and 11 suggesting moderate TBI, as well as a mild traumatic brain injury cohort characterized by GCS scores of 12-15. Blood samples are obtained from each patient on arrival to the emergency department of a hospital within 2 hours of injury and measured by ELISA as described in Examples 1 and 2 for levels of S-100β, UCH-L1, and GFAP in nanograms per milliliter. The results are compared to those of a control group who had not experienced any form of injury. Secondary outcomes include the presence of intracranial lesions in head CT scans. A control group and CT abnormal groups are also studied. Samples are obtained during spinal anesthesia for routine surgical procedures or access to CSF associated with treatment of hydrocephalus or meningitis.

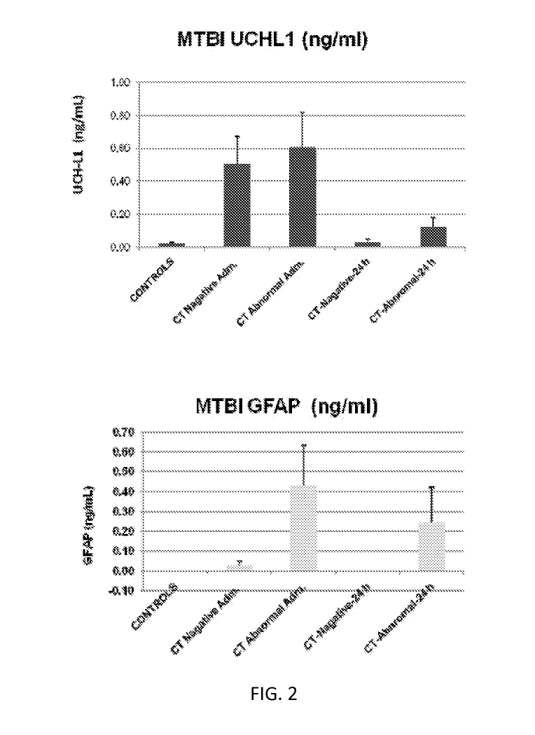

Over 3 months 53 patients are enrolled: 35 with GCS 13-15, 4 with GCS 9-12 and 14 controls. The mean age is 37 years (range 18-69) and 66% were male. The level of biomarkers found in the first available CSF and serum samples obtained and after 24 hours (24 h) in the study are provided in FIGS. 2-4. The quantity of each of the biomarkers of UCH-L1, GFAP, and S-100β are provided for each sample for the cohort of traumatic brain injury sufferers as compared to a control group. Elevated levels of UCH-L1 are indicative of the compromise of neuronal cell body damage while an increase in S-100β suggests recent general trauma, but is aspecific owing the varied body tissues excreting S-100β upon trauma.

The mean GFAP serum level is 0 in control patients, 0.107 (0.012) in patients with GCS 13-15 and 0.366 (0.126) in GCS 9-12 (P<0.001). The difference between GCS 13-15 and controls is significant at P<0.001. In patients with intracranial lesions on CT, GFAP levels are 0.234 (0.055) compared to 0.085 (0.003) in patients without lesions (P<0.001). There is a significant increase in GFAP in serum following a MTBI compared to uninjured controls in both the mild and moderate groups. GFAP is also significantly associated with the presence of intracranial lesions on CT.

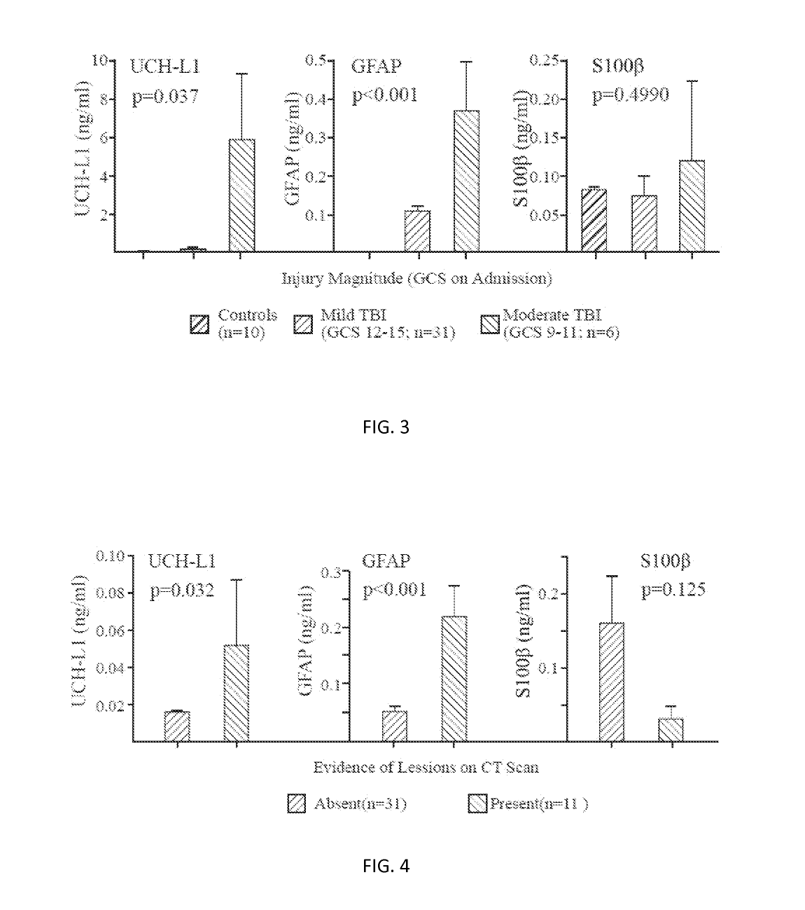

FIG. 2A shows UCH-L1 concentration for controls as well as individuals in the mild/moderate traumatic brain injury cohort as a function of CT scan results upon admission and 24 hours thereafter. FIG. 2B shows GFAP concentration for controls as well as individuals in the mild/moderate traumatic brain injury cohort as a function of CT scan results upon admission and 24 hours thereafter. Simultaneous assays are performed in the course of this study for S-100β biomarkers. The S-100β concentrations are derived from the same samples as those used to determine GFAP and UCH-L1. The concentration of UCH-L1, GFAP, and S100β are provided as a function of injury magnitude between control, mild, and moderate traumatic brain injury as shown in FIG. 3. FIG. 4 shows concentration of the same markers as depicted in FIG. 3 with respect to initial evidence upon hospital admission as a function of lesions observed in tomography scans. Through the simultaneous measurement of S-100β along with GFAP, UCH-L1, or combined with GFAP and UCH-L1 values, rapid and quantifiable determination as to the magnitude of the brain injury is obtained consistent with GSC scoring and CT scanning yet in a more quantifiable, expeditious and economic process.

### Example 4

Controlled cortical impact in vivo model of TBI injury: A controlled cortical impact (CCI) device is used to model TBI on rats essentially as previously described (Pike et al, J Neurochem, 2001 Sep.; 78(6):1297-306, the contents of which are incorporated herein by reference). Adult male (280-300g) Sprague-Dawley rats (Harlan: Indianapolis, Ind.) are anesthetized with 4% isoflurane in a carrier gas of 1:1 O₂/N₂O (4 min) and maintained in 2.5% isoflurane in the same carrier gas. Core body temperature is monitored continuously by a rectal thermistor probe and maintained at 37±1° C. by placing an adjustable temperature controlled heating pad beneath the rats. Animals are mounted in a stereotactic frame in a prone position and secured by ear and incisor bars. Following a midline cranial incision and reflection of the soft tissues, a unilateral (ipsilateral to site of impact) craniotomy (7 mm diameter) is performed adjacent to the central suture, midway between bregma and lambda. The dura mater is kept intact over the cortex. Brain trauma is produced by impacting the right (ipsilateral) cortex with a 5 mm diameter aluminum impactor tip (housed in a pneumatic cylinder) at a velocity of 3.5 m/s with a 1.6 mm compression and 150 ms dwell time. Sham-injured control animals are subjected to identical surgical procedures but do not receive the impact injury. Appropriate pre- and post-injury management is preformed to insure compliance with guidelines set forth by the University of Florida Institutional Animal Care and Use Committee and the National Institutes of Health guidelines detailed in the Guide for the Care and Use of Laboratory Animals. In addition, research is conducted in compliance with the Animal Welfare Act and other federal statutes and regulations relating to animals and experiments involving animals and adhered to principles stated in the “Guide for the Care and Use of Laboratory Animals, NRC Publication, 1996 edition.”

At the appropriate time points (2, 6, 24 hours and 2, 3, 5 days) after injury, animals are anesthetized and immediately sacrificed by decapitation. Brains are quickly removed, rinsed with ice cold PBS and halved. The right hemisphere (cerebrocortex around the impact area and hippocampus) is rapidly dissected, rinsed in ice cold PBS, snap-frozen in liquid nitrogen, and stored at −80° C. until used. For immunohistochemistry, brains are quick frozen in dry ice slurry, sectioned via cryostat (20 μm) onto SUPERFROST PLUS GOLD® (Fisher Scientific) slides, and then stored at −80° C. until used. For the left hemisphere, the same tissue as the right side is collected. For western blot analysis, the brain samples are pulverized with a small mortar and pestle set over dry ice to a fine powder. The pulverized brain tissue powder is then lysed for 90 min at 4° C. in a buffer of 50 mM Tris (pH 7.4), 5 mM EDTA, 1% (v/v) Triton X-100, 1 mM DTT, 1× protease inhibitor cocktail (Roche Biochemicals). The brain lysates are then centrifuged at 15,000×g for 5 min at 4° C. to clear and remove insoluble debris, snap-frozen, and stored at −80° C. until used.

For gel electrophoresis and electroblotting, cleared CSF samples (7 μl) are prepared for sodium dodecyl sulfate-polyacrylamide gel electrophoresis (SDS-PAGE) with a 2× loading buffer containing 0.25 M Tris (pH 6.8), 0.2 M DTT, 8% SDS, 0.02% bromophenol blue, and 20% glycerol in distilled H₂O. Twenty micrograms (20 μg) of protein per lane are routinely resolved by SDS-PAGE on 10-20% Tris/glycine gels (Invitrogen, Cat #EC61352) at 130 V for 2 hours. Following electrophoresis, separated proteins are laterally transferred to polyvinylidene fluoride (PVDF) membranes in a transfer buffer containing 39 mM glycine, 48 mM Tris-HCl (pH 8.3), and 5% methanol at a constant voltage of 20 V for 2 hours at ambient temperature in a semi-dry transfer unit (Bio-Rad). After electro-transfer, the membranes are blocked for 1 hour at ambient temperature in 5% non-fat milk in TBS and 0.05% Tween-2 (TBST) then are incubated with the primary polyclonal UCH-L1 antibody, GFAP antibody, or S-100β antibody in TBST with 5% non-fat milk at 1:2000 dilution at 4° C. overnight. This is followed by three washes with TBST, a 2 hour incubation at ambient temperature with a biotinylated linked secondary antibody (Amersham, Cat #RPN1177v1, for UCH-L1), and a 30 min incubation with Streptavidin-conjugated alkaline phosphatase (BCIP/NBT reagent: KPL, Cat #50-81-08). Molecular weights of intact biomarker proteins are assessed using rainbow colored molecular weight standards (Amersham, Cat #RPN800V). Semi-quantitative evaluation of biomarker protein levels is performed via computer-assisted densitometric scanning (Epson XL3500 scanner) and image analysis with ImageJ software (NIH). UCH-L1 protein is readily detectable by western blot 48 hours after injury at levels above the amounts of UCH-L1 in sham treated and naive samples.

ELISA is used to more rapidly and readily detect and quantitate UCH-L1 in biological samples in rats following CCI. For a UCH-L1 sandwich ELISA (swELISA), 96-well plates are coated with 100 μl/well capture antibody (500 ng/well purified rabbit anti-UCH-L1, made in-house by conventional techniques) in 0.1 M sodium bicarbonate, pH 9.2. Plates are incubated overnight at 4° C., emptied and 300 μl/well blocking buffer (Startingblock T20-TBS) is added and incubated for 30 min at ambient temperature with gentle shaking. This is followed by either the addition of the antigen standard (recombinant UCH-L1) for standard curve (0.05-50 ng/well) or samples (3-10 μl CSF) in sample diluent (total volume 100 μl/well). The plate is incubated for 2 hours at room temperature, then washed with automatic plate washer (5×300 μl/well with wash buffer, TBST). Detection antibody mouse anti-UCH-L1-HRP conjugated (made in-house, 50 μg/ml) in blocking buffer is then added to wells at 100 μL/well and incubated for 1.5 h at room temperature, followed by washing. If amplification is needed, biotinyl-tyramide solution (Perkin Elmer Elast Amplification Kit) is added for 15 min at room temperature, washed then followed by 100 μl/well streptavidin-HRP (1:500) in PBS with 0.02% Tween-20 and 1% BSA for 30 min and then followed by washing. Lastly, the wells are developed with 100 μL/well TMB substrate solution (Ultra-TMB ELISA, Pierce#34028) with incubation times of 5-30 minutes. The signal is read at 652 nm with a 96-well spectrophotometer (Molecular Device Spectramax 190).

UCH-L1 levels of the TBI group (percussive injury) are significantly higher than the sham controls (p<0.01, ANOVA analysis) and the naïve controls as measured by a swELISA demonstrating that UCH-L1 is elevated early in CSF (2h after injury) then declines at around 24 h after injury before rising again 48 h after injury (FIG. 7A).

Similar results are obtained for UCH-L1 in plasma. Blood (3-4 ml) is collected at the end of each experimental period directly from the heart using syringe equipped with 21 gage needle placed in a polypropylene tube. Tubes are centrifuged for 20 min at 3,000×g and the plasma is removed and analyzed by ELISA with results shown in FIG. 7B. UCH-L1 levels of the TBI group are significantly higher than the sham group (p<0.001, ANOVA analysis) and for each time point tested 2 h through 24 h respective to the same sham time points (p<0.005, Student's T-test). UCH-L1 is significantly elevated after injury as early as 2h in serum.

### Example 5

Animal exposure to composite blast: Composite blast experiments are performed using the shock wave generator as described in Svetlov, SI, et al, J. Trauma. 2010 Mar. 2, doi: 10.1097/TA.0b013e3181bbd885, the contents of the entire manuscript of which are incorporated herein by reference.

Rats are anesthetized with 3-5% isoflurane in a carrier gas of oxygen using an induction chamber. At the loss of toe pinch reflex, the anesthetic flow is reduced to 1-3%. A nose cone continues to deliver the anesthetic gases. Isoflurane anesthetized rats are placed into a sterotaxic holder exposing only their head (body-armored setup) or in a holder allowing both head and body exposure. The head is allowed to move freely along the longitudinal axis and placed at the distance 5 cm from the exit nozzle of the shock tube, which is positioned perpendicular to the middle of the head. The head is laid on a flexible mesh surface composed of a thin steel grating to minimize reflection of blast waves and formation of secondary waves that would potentially exacerbate the injury.

For pathomorphology and biomarker studies, animals are subjected to a single blast wave with a mean peak overpressure of 358 kPa at the head, and a total positive pressure phase duration of approximately 10 msec. This impact produces a non-lethal, yet strong effect.

For Analyses of biomarker levels in rat tissues, western blotting is performed on brain tissue samples homogenized on ice in western blot buffer as described previously in detail by Ringger N.C., et al., J Neurotrauma, 2004;21:1443-1456, the contents of which are incorporated herein by reference. Samples are subjected to SDS-polyacrylamide gel electrophoresis and electroblotted onto PVDF membranes. Membranes are blocked in 10 mM Tris, pH 7.5, 100 mM NaCl, and 0.1% Tween-20 containing 5% nonfat dry milk for 60 min at room temperature. Anti-biomarker specific rabbit polyclonal and monoclonal antibodies are produced in the laboratory for use as primary antibodies. After overnight incubation with primary antibodies (1:2,000), proteins are detected using a goat anti-rabbit antibody conjugated to alkaline phosphatase (ALP) (1:10,000-15,000), followed by colorimetric detection system. Bands of interest are normalized by comparison to β-actin expression used as a loading control.

Severe blast exposure in the rat cortex demonstrates no significant increase of GFAP in contrast to a significant GFAP accumulation in hippocampus. GFAP levels peak in hippocampus at 7 day after injury and persist up-to 30 day post-blast.

Quantitative detection of GFAP and UCH-L1 in blood and CSF is determined by commercial sandwich ELISA. UCH-L1 levels are determined using a sandwich ELISA kit from Banyan Biomarkers, Inc., Alachua, Fla. For quantification of glial fibrillary acid protein (GFAP), and neuron specific enolase (NSE) sandwich ELISA kits from BioVendor (Candler, N.C.) are used according to the manufacturer's instructions.

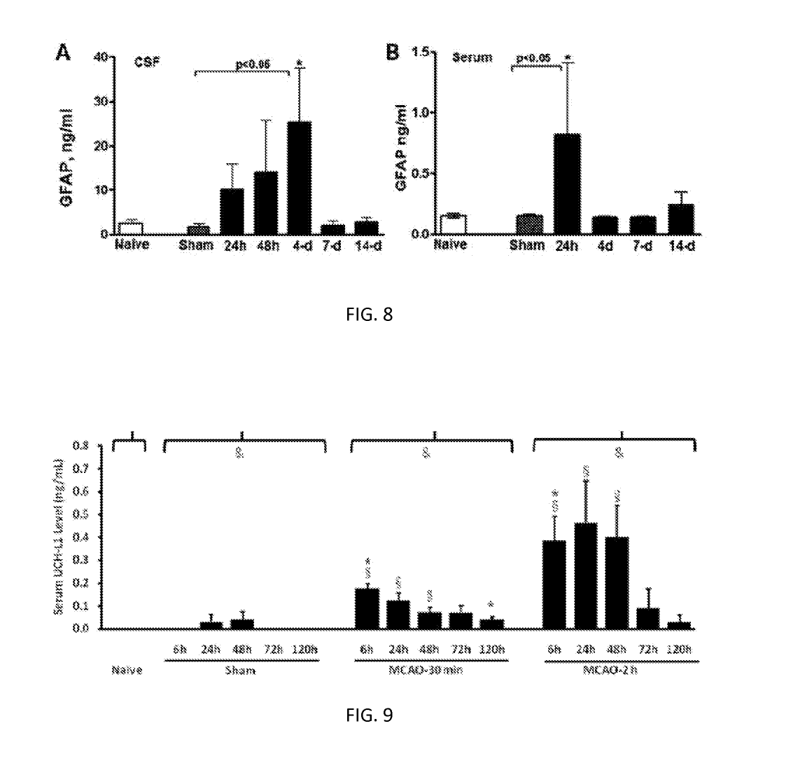

Increase of GFAP expression in brain (hippocampus) is accompanied by rapid and statistically significant accumulation in serum 24 h after injury followed by a decline thereafter. GFAP accumulation in CSF is delayed and occurs more gradually, in a time-dependent fashion (FIG. 8). UCH-L1 levels trend to increased levels in CSF at 24 hours following injury. These levels increase to statistical significance by 48 hours. Plasma levels of UCH-L1 are increased to statistically significant levels by 24 hours followed by a slow decrease.

### Example 6

Screening for neurotoxicity of developmental neurotoxicant compounds. ReNcell CX cells are obtained from Millipore (Temecula, Calif.). Cells frozen at passage 3 are thawed and expanded on laminin-coated T75 cm² tissue culture flasks (Corning, Inc., Corning, N.Y.) in ReNcell NSC Maintenance Medium (Millipore) supplemented with epidermal growth factor (EGF) (20 ng/ml; Millipore) and basic fibroblast growth factor (FGF-2) (20 ng/ml; Millipore). Three to four days after plating (e.g., prior to reaching 80% confluency), cells are passaged by detaching with accutase (Millipore), centrifuging at 300×g for 5 min and resuspending the cell pellet in fresh maintenance media containing EGF and FGF-2. For all experiments, cells are replated in laminin-coated costar 96-well plates (Corning, Inc., Corning, N.Y.) at a density of 10,000 cells per well.

Immunocytochemical experiments and studies of cell media are conducted to determine the level of UCH-L1 and GFAP in cells prior to and following 24 hours of exposure to 1 nM-100 μM of methyl mercury chloride, trans-retinoic acid, D-amphetamine sulfate, cadmium chloride, dexamethasone, lead acetate, 5,5-diphenylhydantoin, and valproic acid essentially as described in Breier JM et al, Toxicological Sciences, 2008; 105(1):119-133, the contents of which are incorporated herein by reference. Cells are fixed with a 4% paraformaldehyde solution and permeabilized in blocking solution (5% normal goat serum, 0.3% Triton X-100 in phosphate-buffered saline). Fluorescein labeled anti-UCH-L1 Antibody #3524 (Cell Signaling Technology, Danvers, Mass.), or GFAP antibody as described in Example 1 is incubated with the fixed cells overnight at 4° C. overnight and visualized using a Nikon TE200 inverted fluorescence microscope with a 20× objective. Images are captured using an RT Slider camera (Model 2.3.1., Diagnostic Instruments, Inc., Sterling Heights, Mich.) and SPOT Advantage software (Version 4.0.9, Diagnostic Instruments, Inc.).

ELISA assays are performed on the cell media of cells following 24 hours of exposure to 1 nM-100 μM of methyl mercury chloride, trans-retinoic acid, D-amphetamine sulfate, cadmium chloride, dexamethasone, lead acetate, 5,5-diphenylhydantoin, and valproic acid essentially as described in Examples 1 and 2 using antibodies to UCH-L1 and GFAP.

### Examples 7-11

Acute oral In vivo drug candidate screening for neurotoxicity. Female Sprague-Dawley rats (Charles River Laboratories, Inc., Wilmington, Mass.) are dosed with methamphetamine (40 mg/kg as four 10 mg/kg intraperitoneal injections (i.p.) (n=8), kainic acid (1.2 nM solution injected i.p.), MPTP (30 mg/kg, s.c.), dizocilpine (0.1 mg/kg, i.p.) or the chemotherapeutic cisplatin (10 mg/kg (single i.p. injection)) (n=4). Anesthesia is performed with intraperitoneal injections of pentobarbital (50 mg/kg). The test substance can also be administered in a single dose by gavage using a stomach tube or a suitable intubation cannula. Animals are fasted prior to dosing. A total of four to eight animals of are used for each dose level investigated.

At 30, 60, 90, and 120 minutes following dosing, the rats are sacrificed by decapitation and blood is obtained by cardiac puncture. The levels of biofluids S-100β, UCH-L1, and GFAP are analyzed by sandwich ELISA or western blot by using biomarker specific antibodies. Relative to control animals, neurotoxic levels of methamphetamine induce increase CSF concentrations of both UCH-L1 and GFAP. Modest increase in S-100b is also observed. Cisplatin, kainic acid, MPTP, and dizocilpine increase UCH-L1, GFAP, and S-100b levels.

### Example 12

Middle cerebral artery occlusion (MCAO) injury model: Rats are incubated under isoflurane anesthesia (5% isoflurane via induction chamber followed by 2% isoflurane via nose cone), the right common carotid artery (CCA) of the rat is exposed at the external and internal carotid artery (ECA and ICA) bifurcation level with a midline neck incision. The ICA is followed rostrally to the pterygopalatine branch and the ECA is ligated and cut at its lingual and maxillary branches. A 3-0 nylon suture is then introduced into the ICA via an incision on the ECA stump (the suture's path was visually monitored through the vessel wall) and advanced through the carotid canal approximately 20 mm from the carotid bifurcation until it becomes lodged in the narrowing of the anterior cerebral artery blocking the origin of the middle cerebral artery. The skin incision is then closed and the endovascular suture left in place for 30 minutes or 2 hours. Afterwards the rat is briefly reanesthetized and the suture filament is retracted to allow reperfusion. For sham MCAO surgeries, the same procedure is followed, but the filament is advanced only 10 mm beyond the internal-external carotid bifurcation and is left in place until the rat is sacrificed. During all surgical procedures, animals are maintained at 37±1° C. by a homeothermic heating blanket (Harvard Apparatus, Holliston, Mass., U.S.A.). At the conclusion of each experiment, if the rat brains show pathologic evidence of subarachnoid hemorrhage upon necropsy they are excluded from the study. Appropriate pre- and post-injury management is preformed to insure compliance with all animal care and use guidelines.

ELISA is used to rapidly and readily detect and quantitate UCH-L1 in biological samples. For a UCH-L1 sandwich ELISA (swELISA), 96-well plates are coated with 100 μl/well capture antibody (500 ng/well purified rabbit anti-UCH-L1, made in-house by conventional techniques) in 0.1 M sodium bicarbonate, pH 9.2. Plates are incubated overnight at 4° C., emptied and 300 μl/well blocking buffer (Startingblock T20-TBS) is added and incubated for 30 min at ambient temperature with gentle shaking. This is followed by either the addition of the antigen standard (recombinant UCH-L1) for standard curve (0.05-50 ng/well) or samples (3-10 μl CSF) in sample diluent (total volume 100 μl/well). The plate is incubated for 2 hours at room temperature, then washed with automatic plate washer (5×300 μl/well with wash buffer, TBST). Detection antibody mouse anti-UCH-L1-HRP conjugated (made in-house, 50 μg/ml) in blocking buffer is then added to wells at 100 μL/well and incubated for 1.5 h at room temperature, followed by washing. If amplification is needed, biotinyl-tyramide solution (Perkin Elmer Elast Amplification Kit) is added for 15 min at room temperature, washed then followed by 100 μl/well streptavidin-HRP (1:500) in PBS with 0.02% Tween-20 and 1% BSA for 30 min and then followed by washing. Lastly, the wells are developed with 100 μl/well TMB substrate solution (Ultra-TMB ELISA, Pierce#34028) with incubation times of 5-30 minutes. The signal is read at 652 nm with a 96-well spectrophotometer (Molecular Device Spectramax 190).

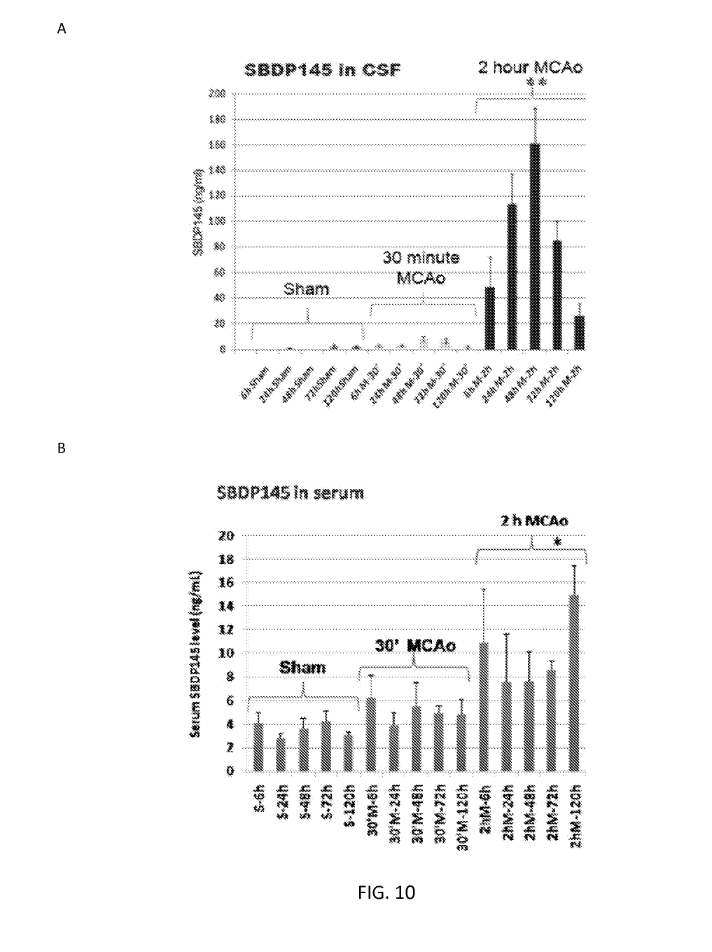

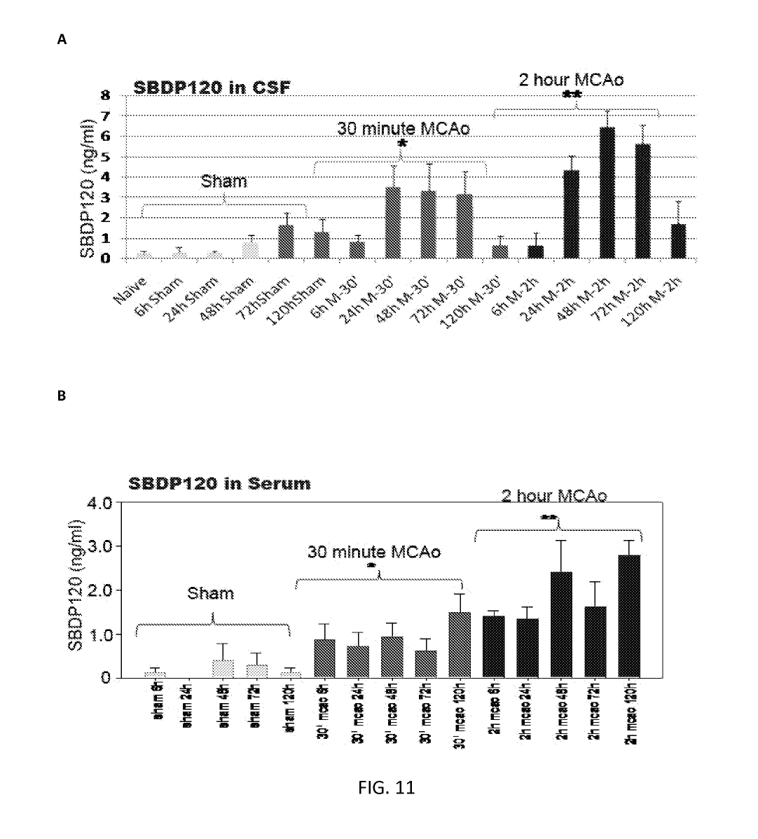

Following MCAO challenge the magnitude of UCH-L1 in serum is dramatically increased with severe (2h) challenge relative to a more mild challenge (30 min). (FIG. 9) The more severe 2 h MCAO group UCH-L1 protein levels are 2-5 fold higher than 30 min MCAO (p<0.01, ANOVA analysis). Group comparison of UCH-L1 levels by ANOVA indicates that all groups at all time points combined (naïve, sham, 30 min MCAO and 2 h MCAO) are significantly different from each other (§ p<0.001). There are also statistically significant differences for 6 h, 24 h, and 48 h time points overall between all groups (& p<0.001). For time points 6 h and 120 h for MCAO-30 min and 6 h for MCAO-2 h, UCH-L1 levels are significantly different from their respective sham time groups (*p<0.05). SBDP145 (FIG. 10) and SBDP120 (FIG. 11) are also significantly increased following MCAO.

### Example 13

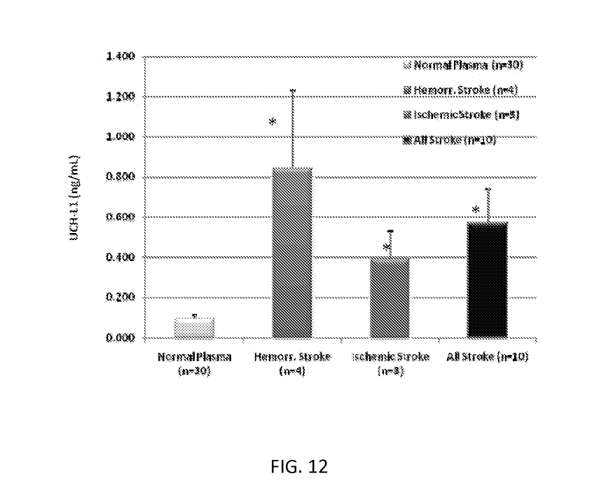

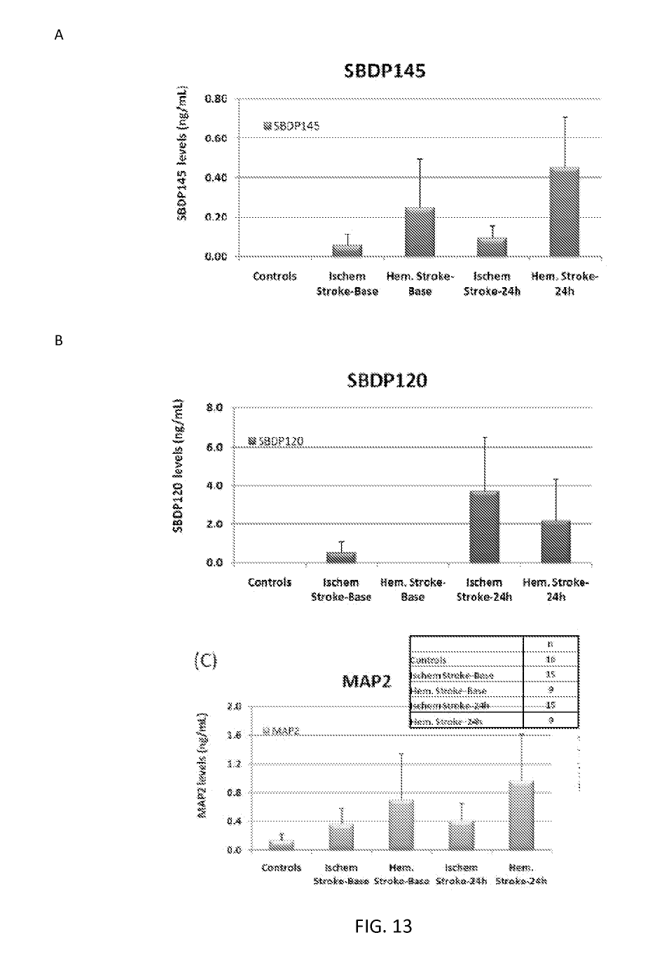

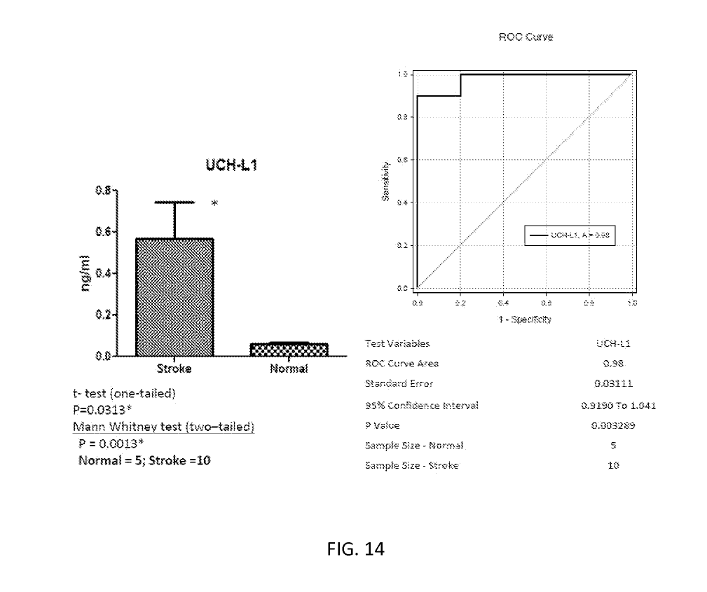

Biomarker levels in biological samples obtained from human stroke patients. Samples of citrated plasma are obtained from blood draws performed within 24 hrs of onset of stroke symptoms of patients (n=10: 5 ischemic stroke, 5 hemorrhagic stroke). UCH-L1 as measured by ELISA as described herein is significantly elevated in blood from stroke patients as compared to normal controls for both hemorrhagic and ischemic groups (FIG. 12). Differences between ischemic and control patients demonstrate a trend P=0.2 but did not reach statistical significance with this small sample set. A preliminary ROC analysis yields a UC of 0.98 (p>0.003). UCH-L1 discriminates between hemorrhagic and ischemic stroke. Levels of SBDP145 and SBDP120 are illustrated in FIGS. 13 A and B respectively.

### Example 14

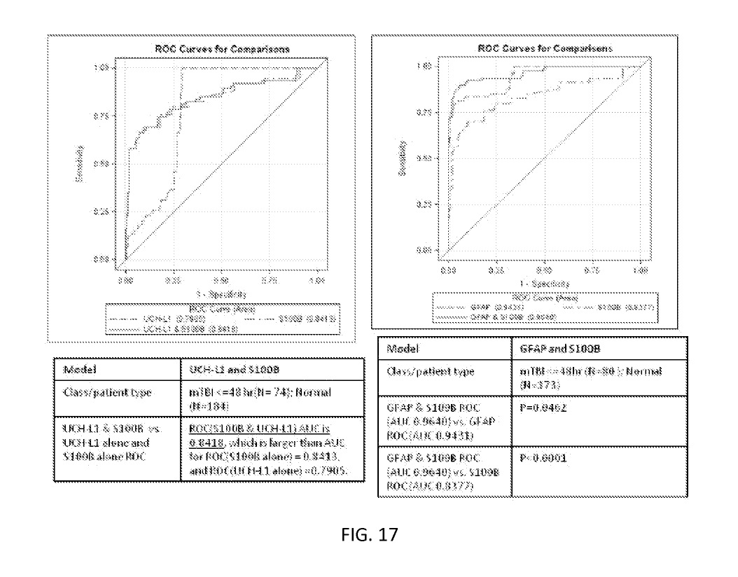

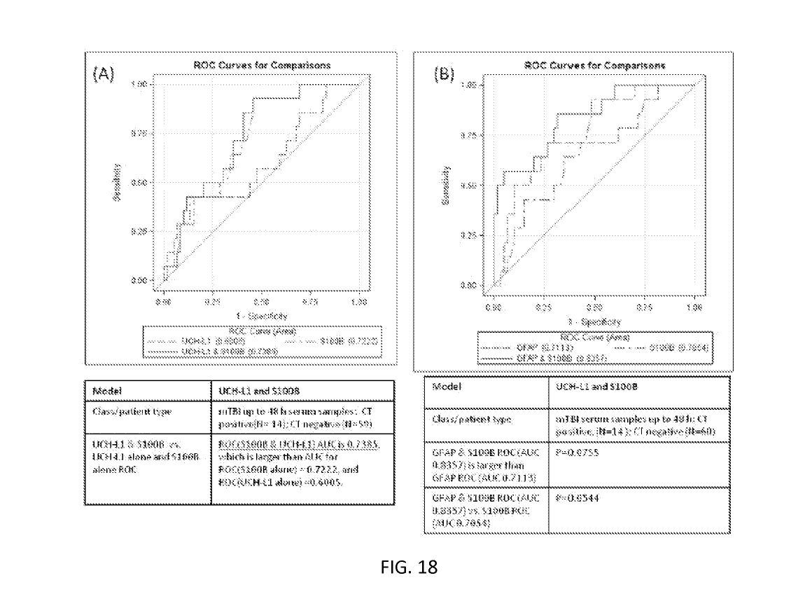

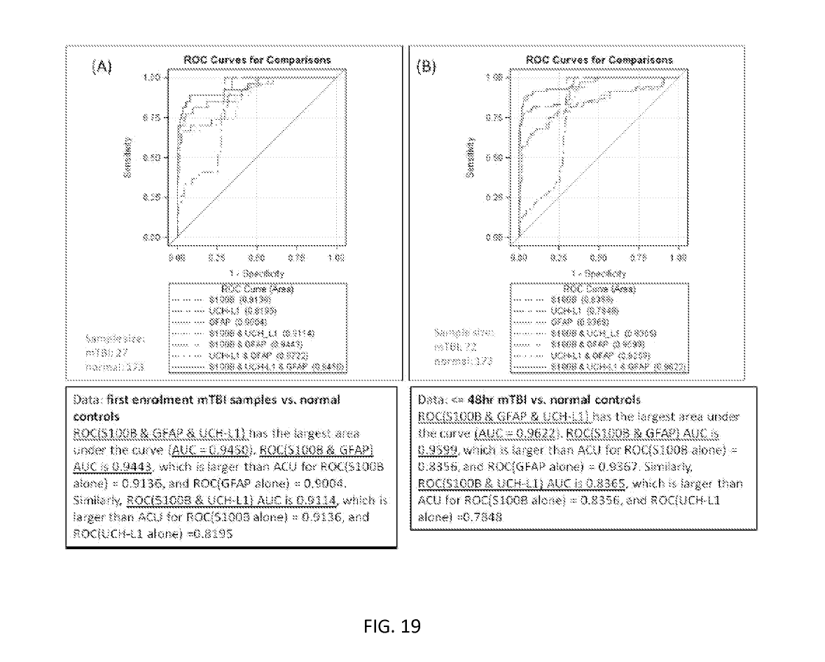

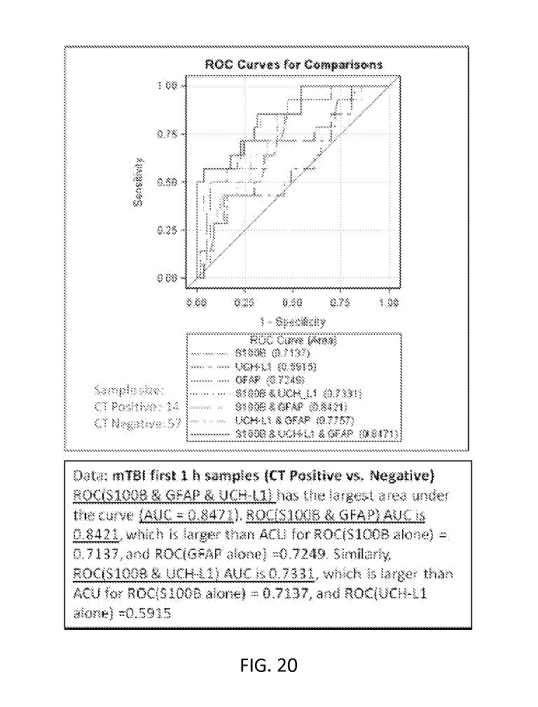

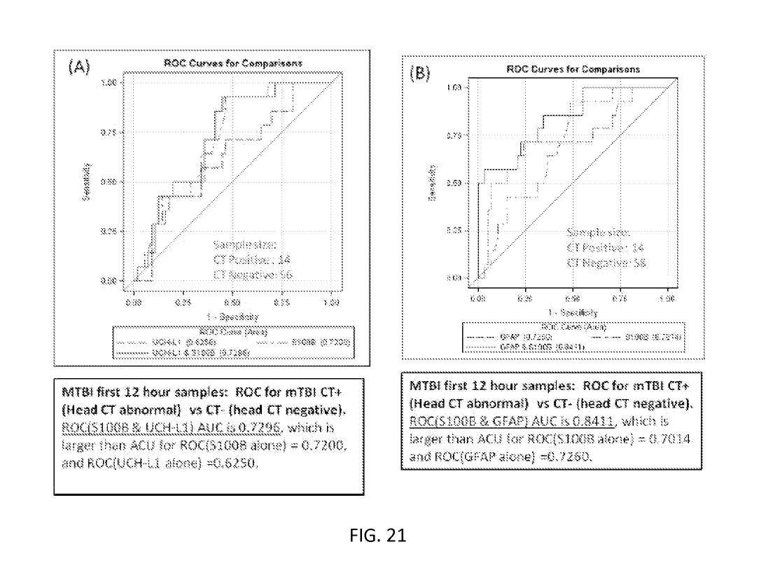

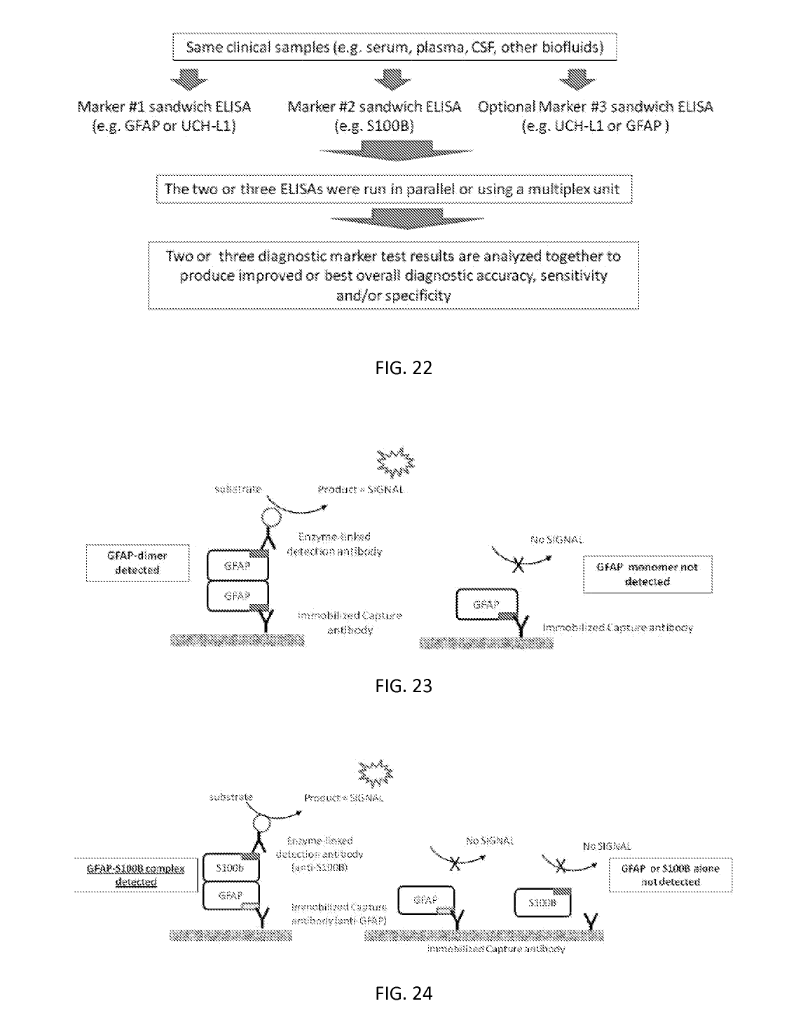

Multiplex assays are performed on human samples of Example 3 where ELISA assays are used to analyze biomarkers S-100b, UCH-L1, and GFAP each alone or in various combinations. The results illustrated in FIGS. 16-21 show that S100b can work together with UCH-L1 and/or GFAP to improve diagnostic accuracy (reflected by area under the curve, or AUC on the Receiver Operating Characteristic (ROC) curve) and improve sensitivity and specificity using mild moderate traumatic brain injury (TBI).

Methods involving conventional biological techniques are described herein. Such techniques are generally known in the art and are described in detail in methodology treatises such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992. The entire contents of each of the aforementioned publications are incorporated herein by reference as if each were explicitly included herein in their entirety.

### REFERENCE LIST

Sorci G, Agneletti A L, Bianchi R, Donato R. Association of S100B with intermediate filaments and microtubules in glial cells. Biochim Biophys Acta. 1998 Dec. 10; 1448(2):277-89. (S100b w GFAP)
Roberta Bianchi, Ileana Giambanco, and Rosario Donato. S-100 Protein, but Not Calmodulin, Binds to the Glial Fibrillary Acidic Protein and Inhibits Its Polymerization in a Ca2+-dependent Manner. J Biol Chem. 1993 Jun. 15; 268(17):12669-74. (S100b w GFAP)
M. Garbuglia, M. Verzini, G. Sorci, R. Bianchi, I. Giambanco, A.L. Agneletti and R. Donato The calcium-modulated proteins, S100A1 and S100B, as potential regulators of the dynamics of type III intermediate filaments. Braz J Med Biol Res. 1999 October; 32(10):1177-85. (S100b w GFAP, Vimentin
Wilhelmsson U, Li L, Pekna M, Berthold C H, Blom S, Eliasson C, Renner O, Bushong E, Ellisman M, Morgan T E, Pekny M. Absence of glial fibrillary acidic protein and vimentin prevents hypertrophy of astrocytic processes and improves post-traumatic regeneration. J. Neurosci. 2004 May 26; 24(21):5016-21. (GFAP-Vimentin)
Lopez-Egido J, Cunningham J, Berg M, Oberg K, Bongcam-Rudloff E, Gobl A. Menin's interaction with glial fibrillary acidic protein and vimentin suggests a role for the intermediate filament network in regulating menin activity. Exp Cell Res. 2002 Aug. 15; 278(2):175-83. (Vimentin-GFAP dimer)
Jing R, Wilhelms son U, Goodwill W, Li L, Pan Y, Pekny M, Skalli O. Synemin is expressed in reactive astrocytes in neurotrauma and interacts differentially with vimentin and GFAP intermediate filament networks. J Cell Sci. 2007 Apr. 1; 120(Pt 7):1267-77. Epub 2007 Mar. 13. (Vimentin-GFAP dimer)
Bheda A, Gullapalli A, Caplow M, Pagano J S, Shackelford J. Ubiquitin editing enzyme UCH L1 and microtubule dynamics: implication in mitosis. Cell Cycle. 2010 Mar. 9(5):980-94. Epub 2010 Mar. 15. (UCH dimer)
Liu Y, Fallon L, Lashuel H A, Liu Z, Lansbury P T Jr. The UCH-L1 gene encodes two opposing enzymatic activities that affect alpha-synuclein degradation and Parkinson's disease susceptibility. Cell. 2002 Oct. 18; 111(2):209-18. (UCH dimer)
Hayes, R. L. Wang, K. K. W., Pike, B. R., (2007) “Detection of Spectrin and Spectrin proteolytic cleavage products in assessing nerve cell damage” U.S. Pat. No. 7,291,710 B2
Hayes, R. L., Wang, K. K. W., Liu, M. C., Oli, M. (2008) “Neural proteins as biomarkers for traumatic brain injury”. U.S. Pat. No. 7,396,654 B2
Hayes, R. L., Wang, K. K. W., Liu, M.C., Oli, M. (2008) “Proteolytic biomarkers for traumatic injury to the nervous system”. U.S. Pat. No. 7,456,027 B2
Raabe and Seifert Neurosurg. Rev. (2000), 23, 3, 136-138

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

The publications referenced are indicative of the levels of those skilled in the art to which the invention pertains. These publications are herein incorporated by reference to the same extent as if each individual publication was specifically and individually indicated to be incorporated by reference.

1. A process for determining the magnitude of traumatic brain injury in a subject comprising:
measuring a quantity a quantity of S-100β in a biological sample obtained from said subject at a first time and contemporaneously measuring a quantity of a second biomarker to determine an extent of traumatic brain injury in said subject.

2. The process of claim 1 wherein said second biomarker is UCH-L1, GFAP, vimentin; SBDP150, SBDP150N, SBDP150i, SBDP145, SBDP120 or MAP2.

3. The process of claim 1 wherein said biological sample is cerebrospinal fluid, whole blood, or a fraction of whole blood.

4. The process of claim 1 wherein said quantity of said second biomarker is measured at the same time as said S-100β.

5. The process of claim 1 further comprising comparing the quantity of said S-100β in said subject to other individuals with no known traumatic brain injury.

6. The process of claim 1 further comprising correlating said quantity of S-100β and said second biomarker with CT scan normality or GCS score.

7. The process of claim 1 wherein said magnitude of brain injury is no traumatic brain injury, mild traumatic brain injury, moderate traumatic brain injury.

8. The process of claim 1 further comprising administering a compound to said subject prior to said measuring.

9. The process of claim 1 wherein said quantity of S-100b and said quantity of said second biomarker are measured in the same biological sample.

10. A process for determining the magnitude of traumatic brain injury in a subject comprising:
measuring a quantity a quantity of S-100β, a quantity of UCH-L1, and a quantity of GFAP in one or more biological samples obtained from said subject at a first time to determine an extent of traumatic brain injury in said subject.

11. The process of claim 10 wherein said biological sample is cerebrospinal fluid, whole blood, or a fraction of whole blood.

12. The process of claim 10 wherein said quantity of said GFAP, UCH-L1 or both are measured at the same time as said S-100β.

13. The process of claim 10 further comprising comparing the quantity of said S-100β, UCH-L1, GFAP or combinations thereof in said subject to other individuals with no known traumatic brain injury.

14. The process of claim 10 further comprising correlating said quantity of S-100β and said quantity of UCH-L1, and said quantity of GFAP with CT scan normality or GCS score.

15. The process of claim 10 wherein said severity of brain injury is no traumatic brain injury, mild traumatic brain injury, or moderate traumatic brain injury.

16. The process of claim 10 further comprising administering a compound to said subject prior to said measuring.

17. The process of claim 10 wherein said quantity of S-100β, UCH-L1 and GFAP are measured in the same biological sample.

18. An assay for determining a magnitude of traumatic brain injury in a subject comprising:
a substrate for holding a sample isolated from the subject;
a S-100β specifically binding agent;
a second biomarker specifically binding agent;
whereby positively reacting said S-100β specifically binding agent and said second biomarker specific binding agent with a portion of the biological sample is evidence of the magnitude of the traumatic brain injury of the subject.

19. The assay of claim 18 further comprising a third biomarker specifically binding agent whereby positively reacting said third biomarker specifically binding agent with a portion of the biological sample is evidence of the magnitude of the traumatic brain injury of the subject.

20. The assay of claim 18 wherein the S-100β specifically binding agent is an antibody.

21. The assay of claim 18 wherein said second biomarker is UCH-L1, GFAP, vimentin; SBDP150, SBDP150N, SBDP150i, SBDP145, SBDP120 or MAP2.

22. The assay of claim 19 wherein said second biomarker is UCH-L1 and said third biomarker is GFAP, vimentin; SBDP150, SBDP150N, SBDP150i, SBDP145, SBDP120 or MAP2.
